(19) United States
(12) Patent Application Publication
(10) Pub. No.: US 2022/0202844 A1
(43) Pub. Date: Jun. 30, 2022

(54) DRUG DELIVERY COMPOSITION AND A METHOD OF ADMINISTERING THE DRUG

(71) Applicant: Richard Clark Kaufman, Santa Monica, CA (US)

(72) Inventor: Richard Clark Kaufman, Santa Monica, CA (US)

(21) Appl. No.: 17/567,759

(22) Filed: Jan. 3, 2022

Related U.S. Application Data
(60) Provisional application No. 63/132,872, filed on Dec. 31, 2020.

(51) Int. Cl. A61K 31/7084; A61K 47/10; A61K 47/12; A61K 47/14; A61K 47/22; A61K 47/26; A61K 9/00

(52) U.S. Cl. CPC A61K 31/7084; A61K 47/10; A61K 47/12; A61K 9/0014; A61K 47/22; A61K 47/26; A61K 47/14

(57) ABSTRACT

A drug delivery system that includes nicotinamide adenine dinucleotide or a derivative or a precursor thereof, and a chemical drug delivery enhancer composition. The drug delivery system enhances the delivery of the nicotinamide adenine dinucleotide or derivatives or precursors thereof through different delivery routes.

### CROSS-REFERENCE TO RELATED APPLICATIONS

This application claims priority from the U.S. provisional patent application Ser. No. 63/132,872, filed on Dec. 31, 2020, which is incorporated herein by reference in its entirety.

### FIELD OF INVENTION

The present invention relates to a composition and method for enhancing delivery of a therapeutic agent, and more particularity, the present invention relates to a composition and method for enhancing delivery of nicotinamide adenine dinucleotide.

### BACKGROUND

The parent pyridine nucleotide, nicotinamide adenine dinucleotide (NAD), is present in all cells of the body. The oxidized form nicotinamide adenine dinucleotide (NAD+) has emerged as a crucial element in bioenergetic and signaling pathways. It is a key regulator of cellular biology, homeostasis, and longevity processes. NAD+ levels steadily decrease with age with resultant alterations in metabolism and increased susceptibility to disease.

NAD+ is required in over 500 enzymatic reactions. It plays a key role in the regulation of almost all major biological processes. NAD+ is a cofactor responsible for electron transport into the respiratory chain. NAD+ and its redox partner, NADH, are central to forming energy-rich ATP molecules within the mitochondria via oxidative phosphorylation of glucose and fatty acids. NAD+ depletion increased spontaneous DNA damage. NAD+ replenishment completely reversed ROS accumulation and DNA damage in skin cells. NAD+ level is critical in preserving the genomic integrity of the cells since DNA repair mechanisms are highly energy dependent. Low NAD+/NADH (oxidized to reduced nicotinamide adenine dinucleotide ratios) promotes cellular senescence, in part by limiting glycolysis and ATP production. Senescent cells display decreased cytosolic NAD+/NADH ratio and increased AMP/ATP and ADP/ATP ratios. NAD+ is required for cellular processes of ATP production, DNA repair, and inflammation that are mediated by PARP-1 and SIRT1 activity. Critical depletion of NAD+ can result in cell death. PARP-1 and SIRT1 may have beneficial or detrimental effects on cell survival depending on the intensity of their activation. Consequently, maintenance of adequate intracellular NAD+ levels and proper SIRT1 and PARP-1 activity is important in preventing DNA damage, genomic instability, chronic inflammation, and cancer.

Skeletal muscle enables posture, breathing, and locomotion. Skeletal muscle also impacts systemic processes such as metabolism, thermoregulation, and immunity. Skeletal muscle is energetically expensive and is a major consumer of glucose and fatty acids. Skeletal muscle cells can contain thousands of mitochondria, which are where the aerobic/oxidative metabolism of glucose occurs. Because NAD+ is required as a hydrogen and electron acceptor and donor for aerobic cellular metabolism, NAD+ localization to mitochondria is important for muscle function. Metabolism of fatty acids and glucose requires NAD+ to function as a hydrogen/electron transfer molecule. Therefore, NAD+ plays a vital role in energy production. Influencing a myriad of cellular processes including mitochondrial biogenesis, transcription, and organization of the extracellular matrix. NAD+ is a major factor in skeletal muscle development, regeneration, aging, and disease.

Studies show that lower NAD+ levels are deleterious for muscle health and higher NAD+ levels enhance muscle health. Increasing the NAD+ levels appear beneficial for multiple types of muscle disease. Given the critical role of NAD+ in muscle development, homeostasis, and aging, Potentiating NAD+ biosynthesis and boosting the NAD+ levels, could be a beneficial adjuvant therapy for a broad spectrum of muscular dystrophies and myopathies.

NAD+ is critical to hydride transfer reactions. It is a precursor of the pyridine nucleotide family, including NADH, NADP+, and NADPH, as well as the end-product of tryptophan metabolism, NAME. The NAD+ co-enzyme family includes regulators of metabolism required for fuel oxidation, ATP generation, gluconeogenesis, and ketogenesis. It is involved in the production of pentose phosphates, heme, lipids, and steroid hormones and helps reduce free radical species. The NAD+ coenzymes drive anabolic reactions, cholesterol and nucleic acid synthesis, elongation of fatty acids, and reduction of oxidized glutathione (GSH).

NAD+ is consumed and may become depleted from reactions controlling DNA repair and nuclear stability, epigenetic control enzymes, situates, intercellular immune communications, and neuronal regeneration. The age-dependent decline in NAD+ levels has consistently been reported due to an imbalance of decreased synthesis and increased consumption of NAD+. Decreased levels of NAD+ are associated with the hallmarks of aging as well as several age-related diseases, including metabolic disorders, cancer, and neurodegenerative diseases. Different conditions contribute to the depletion of NAD+ in the human body. Decreased NAD+ biosynthesis relative NAD+ consumption in metabolic processes; excessive DNA damage due to free radical or ultraviolet attack, resulting in significant poly(ADP-ribose) polymerase (PARP) activation and a high turnover and subsequent depletion of NAD+; Chronic immune activation and inflammatory cytokine production resulting in accelerated CD38 activity.

While multiple clinical and degenerative disorders meet these criteria, chronic accumulation of oxidative stress and inflammation during advanced age represents a major driver of NAD+ decline. Consequentially, increasing NAD+ levels represent a clinically relevant therapeutic strategy to ameliorate age-related decline loss of cellular energy.

Low levels of NAD+ occur in disorders associated with increased oxidative stress/free radical damage such as diabetes, age-related vascular dysfunction, ischemic brain injury, misfolded neuronal proteins, and Alzheimer's dementia. NAD+ depletion may underly a wide range of age-related diseases, such as metabolic disorders, cancer, and neurodegenerative diseases. Emerging evidence implies that elevation of NAD+ levels may slow or even reverse the aspects of aging and also delay the progression of age-related diseases

NAD+ status is critical in preserving genomic stability. Tissues with high cellular turnover, such as skin, require higher doses of NAD+ to counteract genomic insults. Low NAD+ levels increase sun sensitivity in the skin. In animal models, NAD+ deficiency leads to UV sensitivity, impairs DNA damage response, increases genomic instability, and increases skin cancer incidence. NAD+ depletion is associated with human skin aging and cancer. Adequate NAD+ levels are critical for proper DNA repair upon UV exposure. NAD+ deficiency increases skin sensitivity to photo-damage, NAD+ depletion with age and following UV radiation may play a major role in skin cancer initiation by impairing processes involved in genomic stability. Thus, the maintenance of adequate intracellular NAD+ levels is important for skin cancer prevention. Elderly people have lower NAD+ levels and are more prone to developing skin cancer.

Due to NAD+ co-enzymes' major roles in regulating metabolism, energy production, and longevity processes including the benefits of calorie restriction via sirtuins, there is increasing interest in the maintenance of the NAD+ metabolome for antiaging. Augmenting NAD+ levels may improve mitochondrial function and prevent or treat age-associated diseases. NAD+ levels are elevated under conditions of increased life or health span and decline under conditions of accelerated aging or poor health. Supplementation with NAD+ represents a potential therapeutic strategy to mediate protection against the accumulation of inflammation and highly volatile reactive oxygen species (ROS) during aging.

Age-related NAD+ decline is tied to the activation of CD38, a multifunctional ecto-enzyme that metabolizes and mediates NAD+, as well as the NAD+-driven DNA-repair poly-ADP-ribosylation enzymes (PARPs) leading to a further decrease in NAD+ synthesis. CD38 is a cell surface enzyme involved in the degradation of NAD+. Levels and activity of CD38 increase during aging, a cause of the age-related decline in NAD+. CD38 helps regulate metabolic rate, and the pathogenesis of aging, obesity, diabetes, heart disease, asthma, and inflammation. PARPs lead to increased nuclear and cytosolic NAD+ consumption. Inhibitors of CD38 and PARPs are targets for increasing NAD+ levels. Over 200 compounds are listed as CD38 inhibitors in the literature. Natural compounds inhibiting the catalytic activity of CD38 include flavonoids such as apigenin, quercetin, eteolinidin, pterostilbene, and resveratrol.

Boosting NAD+ levels has been shown to extend the lifespan of animals including worms, flies, and rodents. Recent studies have shown that enhancing NAD+ levels can profoundly reduce oxidative cell damage in catabolic tissue, including the brain. IV-infused NAD+ has been used in the treatment of acute and chronic symptoms of substance abuse including alcohol, opiates, cocaine, amphetamines, barbiturates, and other tranquilizers.

Research is ongoing for safe and efficient methods of increasing NAD+ levels especially in the elderly and individuals with chronic diseases. Current methods under investigation for increasing NAD+ include intravenous (IV) infusions; intramuscular (IM) injections; transdermal iontophoretic (IO) patches; oral ingestion of NAD+ precursor molecules; nicotinamide riboside (NR), and nicotinamide mononucleotide (NMN); and inhibitors of CD38 and PARPs.

At present, intravenous infusion of NAD+ is the only proven means of clinically increasing systemic NAD+ levels. NAD+ precursors, including NA, NAM, NMN, NR, and NAR. Replenishment of NAD+ levels via ingestion of NR and NMN, commercial supplements, are slow-acting, and inefficient producing measurable increases in NAD+ levels. Nicotinamide riboside (NR) does not cross the blood-brain barrier, while NAD+ and NADH can do so.

NAD+ is commercially available as a free-flowing powder as well as lyophilized crystals in pharmaceutical purity. NAD+ is heat-labile and unstable at room temperature. At 85° C., thermal degradation of NAD+ results in the generation of nicotinamide and ADP-ribose. Its low 50 mg/ml aqueous solubility limits the ability to obtain therapeutic plasma levels.

A lyophilized NAD+ product formulation is commercially available at 200 mg/ml and is used in intravenous drips and iontophoretic patches. Unfortunately, the freezing of NAD+ in the lyophilization manufacturing process adversely affects the quality of NAD+ through subsequent processing. These impacts result from the low temperature itself, acceleration of degradation reactions, crystallization, product denaturation and aggregation, pH shifts, phase separation, and denaturation of NAD+ at the ice interface. The freezing method and cooling rate during freezing have profound impacts on the morphology and surface area of the final NAD+ product. Lyophilization of NAD+ results in the formation of product crystallization complexes. Making NAD+ as a water-soluble product by lyophilization can inhibit the ability of NAD+ to pass the blood-brain barrier.

Therefore, a need is appreciated for a novel composition and method for delivering NAD+ in therapeutic amounts.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a composition and method for delivering NAD+ in therapeutic amounts.

In one aspect, disclosed is a composition that includes nanostructured nicotinamide adenine dinucleotide and a chemical delivery enhancer.

In one aspect, disclosed is a method that includes the steps of administering the disclosed composition in persons in need thereof, wherein the molecules of nicotinamide adenine dinucleotide can be delivered into the body by different routes and methods of drug delivery including topical, transdermal, sublingual, buccal, nasal, oral, ocular, rectal, injection or injectable, or iontophoretic patch methods of administration.

This disclosure teaches producing synergistic effects between molecules of a nicotinamide adenine dinucleotide composition and a chemical delivery enhancer composition; improving nicotinamide adenine dinucleotide delivery dose concentration and pharmacokinetics from nanostructured nicotinamide adenine dinucleotide formulations; formulating nanostructured nicotinamide adenine dinucleotide dosage forms with increased nicotinamide adenine dinucleotide bioavailability, therapeutic efficacy and bioactivity; manufacturing nanostructured nicotinamide adenine dinucleotide formulations in a unified process; formulating a nanostructured nicotinamide adenine dinucleotide delivery system for increasing dose delivery of nicotinamide adenine dinucleotide and augmenting nicotinamide adenine dinucleotide concentrations; formulating delivery enhancer compositions for co-administration with pharmaceuticals, nutraceuticals, cosmeceuticals, and bioactive compounds; formulating stabile dosage forms of nanostructured nicotinamide-adenine dinucleotides of NAD+, NADH, NADP+, NADPH, or other nicotinamide-adenine dinucleotide derivatives, or analogues; formulating an optimized nanostructured nicotinamide adenine dinucleotide base formulation co-administered with an delivery enhancer formulation containing synergistic ingredients for improving nicotinamide adenine dinucleotide delivery into the bloodstream by different drug delivery routes of administration; formulating chemical delivery enhancers for co-administration with chemical and nutraceutical inhibitors of CD38 and, PARPs to increase nicotinamide adenine dinucleotide levels; and augmenting nicotinamide adenine dinucleotide levels with a nanostructured nicotinamide adenine dinucleotide delivery system for increasing nicotinamide adenine dinucleotide blood concentrations, improving the nicotinamide adenine dinucleotide metabolome, improve mitochondrial function, increasing ATP generation, treating age-associated diseases, improving muscular performance and treating degenerative conditions.

Disclosed are the compositions and methods of a nicotinamide adenine dinucleotide delivery system comprised of two compositions: a nanostructured nicotinamide adenine dinucleotide dosage form and a chemical delivery enhancer dosage form that are co-administered to the body.

Disclosed is a drug delivery system that includes a co-administered nanostructured nicotinamide adenine dinucleotide composition and a chemical delivery enhancer composition, wherein the drug delivery system can be administered to the body by different routes drug delivery including topical, transdermal, intraoral, sublingual, buccal, intranasal, oral, ocular, rectal, injection, and iontophoretic patch administration.

Disclosed are the compositions and methods of a nanostructured nicotinamide adenine dinucleotide drug delivery system comprised of two formulations: a nanostructured nicotinamide adenine dinucleotide composition and a chemical delivery enhancer composition, co-administering them to the bloodstream as a non-invasive method of augmenting nicotinamide adenine dinucleotide levels.

Disclosed are the compositions and methods of a nanostructured nicotinamide adenine dinucleotide drug delivery system comprised of two formulations: a nanostructured nicotinamide adenine dinucleotide composition and a chemical delivery enhancer composition, the method includes the steps of co-administering them to the body by different non-invasive drug delivery routes and methods for therapeutic applications.

Disclosed are the compositions and methods of a nanostructured nicotinamide adenine dinucleotide drug delivery system that co-administers two product compositions: a dosage form of a nanostructured nicotinamide adenine dinucleotide composition along with a dosage form of chemical delivery enhancer composition that are combined before entering the body.

Disclosed are the compositions, methods of use, and formulations of nanostructured nicotinamide adenine dinucleotides dosage forms for nicotinamide-adenine dinucleotide therapy.

Disclosed are the compositions and methods of formulating a nanostructured nicotinamide adenine dinucleotide composition for delivering a therapeutic dose of nicotinamide adenine dinucleotide to the body by different methods of administration that include topical, transdermal, sublingual, buccal, nasal, oral, ocular, rectal, injection, inhalation, and iontophoretic patch methods of administration.

Disclosed are the methods of manufacturing the stabilized bioidentical nicotinamide adenine dinucleotide that has not undergone lyophilization and structural processing.

Disclosed are the compositions of nanostructured nicotinamide adenine dinucleotide dosage forms for improving the pharmacokinetic characteristics of NAD+ including area under the curve (AUC), plasma concentration-time profile, maximum plasma concentration (Cmax), time to maximum plasma concentration (Tmax), and volume of distribution (Vd or Vss), elimination half-life (t½), and clearance (CL).

Disclosed are the compositions and methods of formulating and administering stable dosage forms of nanostructured nicotinamide-adenine dinucleotides of NAD+ (oxidized nicotinamide-adenine dinucleotide), or NADH (reduced nicotinamide-adenine dinucleotide), or NADP+ (oxidized nicotinamide-adenine dinucleotide phosphate), or NADPH (reduced nicotinamide-adenine dinucleotide phosphate), or other nicotinamide-adenine dinucleotide derivatives or analogs into the body.

Disclosed are the compositions and methods of manufacturing stable dosage forms nicotinamide adenine dinucleotide for intraoral, oral, gastrointestinal, transdermal, nasal, ocular, rectal, or vaginal, injection routes of administration.

Disclosed are the compositions and methods of formulating nanostructured nicotinamide adenine dinucleotide dosage forms comprised of molecules with increased bioavailability, bioactivity, and therapeutic activity.

Disclosed are the compositions and methods of formulating and manufacturing dosage forms of a chemical drug delivery enhancer for nicotinamide adenine dinucleotide.

Disclosed is a chemical drug delivery enhancer for improving the delivery or transport molecules of nicotinamide adenine dinucleotide into the systemic circulation by different routes and methods of administration that include topical, transdermal, sublingual, buccal, nasal, oral, ocular, rectal, injection, inhalation, and iontophoretic patch methods of administration.

Disclosed are the methods of drug delivery enhancer dosage forms for nicotinamide adenine dinucleotide that incorporate bioavailability enhancers to increase nicotinamide adenine dinucleotide dosage delivery into a human body.

Disclosed are the compositions and methods for producing synergistic effects between molecules of a chemical drug delivery enhancer and a dosage form of nanostructured nicotinamide adenine dinucleotide for improving delivery characteristics of nicotinamide adenine dinucleotide.

Disclosed are the compositions and methods of formulating bioavailability enhancers of nicotinamide adenine dinucleotide for increasing nicotinamide adenine dinucleotide bioavailability, bio-efficacy, and reducing therapeutic dosages.

Disclosed are the methods and compositions of formulating a nano-structured nicotinamide adenine dinucleotide drug delivery system that combines nicotinamide adenine dinucleotide with a chemical drug delivery enhancer before administration.

Disclosed are the compositions and methods of formulating chemical drug delivery enhancers for co-administration with inhibitor molecules of CD38 and PARPs to increase nicotinamide adenine dinucleotide levels.

Disclosed are the compositions and methods for formulating a chemical delivery enhancer for co-administration with apigenin, quercetin, resveratrol, or pterostilbene, and delivering them in the body by different routes of administration and delivering them into the body with increased bioavailability to increase nicotinamide adenine dinucleotide levels.

Disclosed are the compositions and methods for formulating a nanostructured nicotinamide adenine dinucleotide drug delivery system producing synergistic effects between molecules of nanostructured nicotinamide adenine dinucleotide and molecules of a chemical delivery enhancer composition as a result of their co-administration.

Disclosed are the compositions and methods of formulating a nanostructured nicotinamide adenine dinucleotide drug delivery system producing synergistic effects between molecules of nanostructured nicotinamide adenine dinucleotide and molecules of a chemical delivery enhancer composition for augmenting endogenous nicotinamide adenine dinucleotide levels in the body.

Disclosed are the compositions and methods of transdermal delivery of nanostructured nicotinamide adenine dinucleotide dosage forms in delivery vehicles through the skin to systemic circulation as the target of nicotinamide adenine dinucleotide. Whereas the nicotinamide adenine dinucleotide penetrates through the skin's protective stratum corneum into the dermal layers for systemic absorption via the dermal microcirculation.

Disclosed are the compositions and methods of transdermal delivery of nanostructured nicotinamide adenine dinucleotide as a non-invasive alternative to parenteral routes of nicotinamide adenine dinucleotide administration.

Disclosed are the compositions and methods of transdermal delivery of nanostructured nicotinamide adenine dinucleotide providing increased bioavailability and improved pharmacokinetic profiles of nicotinamide adenine dinucleotide.

Disclosed are the compositions and methods of intranasal delivery of nanostructured nicotinamide adenine dinucleotide dosage forms in delivery vehicles through the nasal mucosa to systemic circulation as the target of nicotinamide adenine dinucleotide.

Disclosed are the compositions and methods of intranasal delivery of nanostructured nicotinamide adenine dinucleotide as a non-invasive alternative to parenteral routes of nicotinamide adenine dinucleotide administration.

Disclosed are the compositions and methods for intranasal delivery of nanostructured nicotinamide adenine dinucleotide providing increased bioavailability and improved pharmacokinetic profiles of nicotinamide adenine dinucleotide.

Disclosed are the compositions and methods for intraoral nicotinamide adenine dinucleotide drug delivery.

Disclosed are the compositions and methods of intraoral delivery of nanostructured nicotinamide adenine dinucleotide dosage forms in delivery vehicles through the sublingual or buccal mucosa to the systemic circulation as the target of nicotinamide adenine dinucleotide.

Disclosed are the compositions and methods of intraoral delivery of nanostructured nicotinamide adenine dinucleotide as a non-invasive alternative to parenteral routes of nicotinamide adenine dinucleotide administration.

Disclosed are the compositions and methods of intraoral delivery of nanostructured nicotinamide adenine dinucleotide providing increased bioavailability and improved pharmacokinetic profiles of nicotinamide adenine dinucleotide.

Disclosed are the compositions and methods of oral delivery of nanostructured nicotinamide adenine dinucleotide dosage forms in delivery vehicles through the lumen of the gastrointestinal to systemic circulation as the target of nicotinamide adenine dinucleotide.

Disclosed are the compositions and methods of oral delivery of nanostructured nicotinamide adenine dinucleotide as a non-invasive alternative to parenteral routes of nicotinamide adenine dinucleotide administration.

Disclosed are the compositions and methods of oral delivery of nanostructured nicotinamide adenine dinucleotide providing increased bioavailability and improved pharmacokinetic profiles of nicotinamide adenine dinucleotide

Disclosed are the compositions and methods for nanostructured nicotinamide adenine dinucleotide for therapy.

Disclosed are the compositions and methods of a nanostructured nicotinamide adenine dinucleotide drug delivery system for regulating metabolism, energy production, or longevity processes.

Disclosed are the compositions and methods of a nanostructured nicotinamide adenine dinucleotide drug delivery system for benefits of calorie restriction via sirtuins or maintenance of the NAD+ metabolome.

Disclosed are the compositions and methods of nanostructured nicotinamide adenine dinucleotide drug delivery system for augmenting nicotinamide adenine dinucleotide levels for improving mitochondrial function, preventing, or treating age-associated diseases.

Disclosed are the compositions and methods for nanostructured nicotinamide adenine dinucleotide drug delivery system for therapeutic applications including NAD+ replacement, improving the NAD+ metabolome, increasing ATP regeneration, or treating aging disorders.

Disclosed are the compositions and methods of formulating stable dosage forms of nanostructured nicotinamide-adenine dinucleotides of NAD+ (oxidized nicotinamide-adenine dinucleotide), NADH (reduced nicotinamide-adenine dinucleotide), NADP+ (oxidized nicotinamide-adenine dinucleotide phosphate, NADPH (reduced nicotinamide-adenine dinucleotide phosphate), other nicotinamide-adenine dinucleotide derivatives, or nicotinamide-adenine dinucleotide analogs.

Disclosed are the compositions and methods of formulating stable dosage forms of nicotinamide adenine dinucleotide using lyophilized nicotinamide adenine dinucleotide or non-lyophilized nicotinamide adenine dinucleotide.

Disclosed are the compositions and methods of formulating stable dosage forms of nicotinamide adenine dinucleotide, and nanostructured nicotinamide adenine dinucleotide by incorporating solvents that such as glycol in a drug delivery vehicle.

Disclosed are the compositions and methods for formulating stable dosage forms of nicotinamide adenine dinucleotide or nanostructured nicotinamide adenine dinucleotide by incorporating phospholipids in a drug delivery vehicle.

Disclosed are the compositions and methods of formulating stable dosage forms of nicotinamide adenine dinucleotide or nanostructured nicotinamide adenine dinucleotide by incorporating polymers in a drug delivery vehicle.

Disclosed are the compositions and methods for formulating stable dosage forms of nicotinamide adenine dinucleotide or nanostructured nicotinamide adenine dinucleotide by incorporating buffers in a drug delivery vehicle.

Disclosed are the compositions and methods of formulating stable dosage forms nicotinamide adenine dinucleotide, nanostructured nicotinamide adenine dinucleotide for therapy that prevents physiochemical degradation of nicotinamide adenine dinucleotide with a stabilizer or protective molecular structure.

### DETAILED DESCRIPTION

The term “NAD” in the present disclosure refers to the parent pyridine, nucleotide, nicotinamide adenine dinucleotide.

The term “NAD+” in the present disclosure refers to oxidized nicotinamide-adenine dinucleotide and NAD+ dosage forms sold as powders and lyophilized powders with increase water solubility and emulsion ability.

The term “NADH” in the present disclosure refers to reduced nicotinamide-adenine dinucleotide.

The term “NADP+” in the present disclosure refers to oxidized nicotinamide-adenine dinucleotide phosphate.

The term “NADPH” in the present disclosure refers to reduced nicotinamide-adenine dinucleotide phosphate.

The term “BioNAD+” and “Bioidentical NAD+” in the present disclosure refer to the oxidized dosage forms of NAD+ (oxidized nicotinamide-adenine dinucleotide) with a CAS #53-84-9 and as raw powders that have not undergone lyophilization, freeze-drying, or additional processing; and are structural or functionally identical or equivalent to the endogenous NAD+ manufactured by the human and animal body.

The term “lyophilized nicotinamide adenine dinucleotide” in the present disclosure refers to nicotinamide adenine dinucleotide molecules that have undergone a lyophilization for increased water solubility; and is available in crystalline or powder forms for reconstitution, and administered in NAD+ intravenous drips, injections, or Iontophoretic patches.

The term “nanostructured nicotinamide adenine dinucleotide” in the present disclosure refers to nanosized molecular structures of nicotinamide adenine dinucleotide containing molecules in a nanometer size dimension.

The term “drug delivery system” in the present disclosure refers to the system as a medium or carrier for administering a therapeutic agent or drug to a patient's body.

The term “nanostructured nicotinamide adenine dinucleotide drug delivery system” in the present disclosure refers to compositions of nanostructured nicotinamide adenine dinucleotide and a chemical drug delivery enhancer, encompassing protocols of co-administered dosage forms of nicotinamide adenine dinucleotide, or nanostructured nicotinamide adenine dinucleotide, or nicotinamide adenine dinucleotide analogs, or nicotinamide adenine dinucleotide derivatives with a drug delivery enhancer.

The term “drug delivery systems” (DDSs) in the present disclosure refers pharmaceutical formulations, engineered technologies, or devices for the targeted delivery and/or controlled release nicotinamide adenine dinucleotide and therapeutic agents in our body.

Following administration, the DDSs liberates nicotinamide adenine dinucleotide and their active ingredients, and subsequently, the bioactive molecules are transported across various biological barriers to reach the site of action.

Drug delivery systems are engineered technologies for the targeted delivery and/or controlled release of therapeutic agents and can be described in four broad categories: routes of delivery, delivery vehicles, cargo, and targeting strategies.

The term “drug delivery vehicle” in the present disclosure refer to a drug delivery system that is used as a medium or “carrier” for administering nicotinamide adenine dinucleotide, or a therapeutic agent or drug to a body.

The term “drug delivery cargo” in the present disclosure refers to nicotinamide adenine dinucleotide, or a therapeutic agent, or drug delivered by a drug delivery system.

The term “nanostructured delivery system” in the present disclosure refers to drug delivery vehicles in which at least one dimension is in the range of nanometer-scale (or composed of them as basic units in three-dimensional space) formulated to deliver a drug or phytochemical or molecule or compound into the body or brain or targeted sites in a controlled manner.

A list of nanostructured drug delivery systems includes polymer-based, non-polymeric, and lipid-based nanostructured drug delivery systems, particles, and emulsions.

Nanostructured drug delivery systems also include dendrimers, solid lipid nanoparticles, nanostructured lipid compositions, micelles, reverse micelles, nanogels, protein nanoparticles, carbon nanotubes, metallic nanoparticles, quantum dots, silica-based nanoparticles, liposomes, ethosomes, nanospheres, hydrogels, organogels, lipid-polymer nano matrixes, nano emulsions, and self-nanoemulsifying drug delivery systems.

The terms “dosage form”, “drug dosage form” in the present disclosure refer to the physical form of a dose of a chemical compound used as a drug, medication, nutraceutical, cosmeceutical, or dietary intended for administration or consumption. Dosage forms include pills, tablets, capsules, oral suspensions, tinctures, emulsions, liquids drinks, powders, gels, creams, lotions, liniments, sprays, suppositories, crystals, aerosols, vaporization, liquid injections, transdermal liquids, gels, patches, eye drops, eye gels, eye ointments, nasal sprays, nasal gels, nasal solutions, oral strips, mucoadhesive buccal or sublingual compositions, and among many others in the present disclosure. The route of administration delivery is dependent on the dosage form of the substance.

The term “CD38” in the present disclosure refers to “Cluster of Differentiation 38”, a multifunctional ecto-enzyme that metabolizes nicotinamide adenine dinucleotide and mediates nicotinamide dinucleotide (NAD+) and extracellular nucleotide homeostasis as well as intracellular calcium. Of greatest significance for this disclosure is the role of CD38 as an ecto-enzyme capable of modulating extracellular nicotinamide adenine dinucleotide precursor availability. Inhibitors of the catalytic activity of CD38 lead to an increase in cellular NAD+ levels and activation of nicotinamide adenine dinucleotide-dependent enzymes such as SIRTUINs.

The term “CD38 inhibitors” in the present disclosure refers to molecule inhibitors of the catalytic activity of CD38 that can boost cellular NAD+ levels and activate NAD-dependent enzymes such as SIRTUINs that show promise as nicotinamide adenine dinucleotide boosting drugs.

CD38 inhibitors refer to over 200 compounds capable of inhibiting CD38 classified as nicotinamide adenine dinucleotide analogs flavonoids, and heterocyclic compounds that bind to amino acids located in the active site of CD38. It refers to natural CD38 inhibitor compounds or molecules that include apigenin, quercetin, leteolinidin, resveratrol, or pterostilbene.

The term “stratum corneum” in the present disclosure refers to the external layer of the epidermis. The epidermis is the outermost layer, which consists primarily of layers of keratinized epithelium, under which lies the dermis, a layer of connective tissue that contains a rich network of the blood and lymph vessels, hair follicles, and sweat and sebaceous glands.

The term “transdermal drug delivery” in the present disclosure refers to delivering dosage forms of nicotinamide adenine dinucleotide or drugs systemically by applying formulation onto intact or healthy skin. Nicotinamide adenine dinucleotide or other active compounds penetrate through the stratum corneum and pass through the deeper epidermis to the dermal layer where nicotinamide adenine dinucleotide or other compounds become available for systemic absorption via the dermal microcirculation.

The term “topical drug delivery” in the present disclosure refers to delivering dosage forms of nicotinamide adenine dinucleotide, drugs, or a substance to the skin, where the skin is itself the target of the active substance.

The term “intradermal drug delivery” in the present disclosure refers to delivering dosage forms of nicotinamide adenine dinucleotide, drugs, or a substance across the stratum corneum to epidermal and dermal layers that are the target of the active substance.

The term “intraoral drug delivery” in the present disclosure refers to delivering dosage forms of nicotinamide adenine dinucleotide or drugs, or substances from the oral cavity by buccal and sublingual methods of delivery.

The term “buccal delivery” in the present disclosure refers to delivering nicotinamide adenine dinucleotide, drugs, or substances across the buccal mucosa (the epithelium lining of the cheeks, gums, and lips) into the bloodstream. Effective buccal delivery systems require a mucoadhesive agent to increase mucosal retention time and absorption.

The term “sublingual delivery” in the present disclosure refers to delivering nicotinamide adenine dinucleotide, drugs, or substances under the tongue into the bloodstream. Effective sublingual delivery systems require a mucoadhesive agent to increase mucosal retention time and absorption before salivary washout carries the active ingredients down your throat.

The terms “intranasal delivery” in the present disclosure refer to delivery systems designed to delivering nicotinamide adenine dinucleotide drugs and substances across nasal mucosa into the bloodstream. Intranasal delivery includes direct nose-to-brain delivery systems along the olfactory and trigeminal that bypass the restrictive blood-brain barrier preventing most substances from entering the brain. Effective intranasal delivery systems require a mucoadhesive agent to increase mucosal retention time, absorption, and prevent ciliary motion carrying active ingredients down your throat.

The terms “gastrointestinal delivery” and “oral delivery” in the present disclosure refer to delivering nicotinamide adenine dinucleotide, drugs, or substances down your throat to the intestinal mucosa for absorption into the bloodstream. The oral route is by far the most common route of drug administration.

The terms “co-administering”, “co-administration” and “concomitant drugs” in the present disclosure refer to two or more drugs or nicotinamide adenine dinucleotide, and a chemical drug delivery enhancer composition administered either at the same time, or almost the same time, or in sequence.

The term “drug delivery enhancer” in the present disclosure refers to chemical agents or physical methods used to deliver or transport micro and macromolecules of nicotinamide adenine dinucleotide, drugs, other compounds across the skin, or other cell membranes that may increase their bioavailability or bioactivity.

Examples of chemical drug delivery enhancer agent in the present disclosure include sulphoxides, amines, amides, azones, pyrrolidones, alcohols, alkanols surfactants, solvents, glycols, fatty acids, acidifiers, lipids, terpenes & terpenoids, cyclodextrins, or vesicles. It also refers to their combinations as vesicles, colloids, microemulsions, eutectic mixtures, inclusion complexes, or nano-dimensional structures.

The term “drug delivery enhancer” in the present disclosure refers to physical methods that deliver or transport molecules of nicotinamide adenine dinucleotide, or other substances into the body, or across the skin, or across cell membranes that include iontophoresis, electroporation, sonophoresis, laser radiation and radiofrequency, microneedles, or ablation.

The term “bioavailability enhancer” in the present disclosure refers to agents, chemicals, compounds, and molecules incorporated into chemical drug delivery enhancers which increase the physiological availability of a given amount of nicotinamide adenine dinucleotide, a drug, or chemical of its administered dose that is absorbed into the bloodstream; and increase delivery across the cell membranes, potentiating their molecular conformational interaction, acting as receptors for their molecule, and making target cells more receptive to them.

The term bioavailability enhancer in the present disclosure includes agents, chemicals, compounds, or molecules for improving the transport of nicotinamide adenine dinucleotide, drugs, and substances across the intestinal epithelium, through the skin, across the blood-brain barrier, across sublingual mucosa, across buccal mucosa, across nasal mucosa, or across a cell membrane, and increases nicotinamide adenine dinucleotide, or drugs bioavailability.

The term “delivery” is the present disclosure refers to the introduction of nicotinamide adenine dinucleotide, a molecule, a compound, or into a cell.

The terms “synergistic effects” or “synergy” in the present disclosure refer to additive effects in the domains of pharmacology and biomedicine, where the effect of two or more agents, chemicals or drugs working in combination have greater dose responses and effects than the expected result of said single agents alone. The effects between two or more molecules of chemical delivery enhancers are greater than a single chemical delivery enhancer agent.

Molecules of chemical delivery enhancer compositions are additive to molecules of nicotinamide adenine dinucleotide compositions from co-administration in a nicotinamide adenine dinucleotide drug delivery system.

The term “nicotinamide adenine dinucleotide therapy” in the present disclosure refers to nanostructured nicotinamide adenine dinucleotide delivery or other methods of raising endogenous nicotinamide adenine dinucleotide levels for therapeutic applications. This includes potential uses with conditions responsive to nicotinamide adenine dinucleotide such as neurodegenerative diseases, Alzheimer's, Parkinson's, degenerative aging disorders, chronic fatigue syndrome, high cholesterol, depression, high blood pressure, and substance abuse. Potential use for conditions of age-dependent decline in NAD+ and potential treatment of DNA damage, epigenetic alteration, deregulated nutrient-sensing, loss of proteostasis, altered cellular communication, cellular senescence, stem cell exhaustion, mitochondrial dysfunction, compromised autophagy, telomere attrition, DNA repair, epigenetically modulated gene expression, maintenance of intracellular calcium homeostasis, and immune disorders; or treatments of the NAD+ metabolome and treatments for increasing ATP.

The term “chemical stabilizer” in the present disclosure refers to a chemical formulation, compound, or molecule to prevent drug degradation, and/or to create stability of nicotinamide adenine dinucleotide molecules and nanostructures. They are components and ingredients in the formulations, nano-dimensional drug delivery systems, nano-dimensional structures, formulations, and chemical drug delivery enhancers of this disclosure. Examples of classes of the chemical stabilizer include antioxidants, chelators, spin traps, acidifiers, ionic pairs, preservatives, and physical barriers coatings.

The term “drug stability” in the present disclosure refers to the ability of nicotinamide adenine dinucleotide or a drug dosage form to maintain physical, chemical, therapeutic, and microbial properties during the time of storage and patient use.

The term “degradation” in the present disclosure refers to the incapacity or incapability of nicotinamide adenine dinucleotide, or any drug, compound, or any formulation to remain within a particular chemical, microbiological, therapeutical, physical, and toxicological specification.

The term “liquid dosage form” in the present disclosure refers to a solution, suspension, multiphase dispersion, syrup, gel, emulsion, nano emulsion, liquid nanostructure, reconstituted powder, liquid preparation, liquid composition, liquid drug formulation, and liquid drug preparation.

The term “physiochemical” in the present disclosure refers to both physical and chemical properties, changes, and reactions. The ability of a chemical compound to elicit a pharmacological/therapeutic effect is related to the influence of various physical and chemical (physicochemical) properties of the chemical substance on the biomolecule with which it interacts.

The term “dispersions” in the present disclosure refers to multi-phase mixtures consisting of substances that are insoluble in one another. One phase is the continuous phase in which small particles of the other phase are dispersed.

The term “bioactive substance” in the present disclosure refers to a substance or compound or molecules having an effect on, or causing a reaction, or triggering a response in living tissue or on a living organism, presenting therapeutic potential.

The term “route of administration” in the present disclosure refers to the path or delivery route or location by which nicotinamide adenine dinucleotide, a drug, molecule or compound, or other substance is taken into the body. Routes of administration refer to oral, gastrointestinal intraoral (sublingual, buccal), transdermal, topical, nasal, intranasal, inhalation, nebulization, vaporization, ocular, vaginal, rectal, injection (intravenous, intramuscular, subcutaneous), transmucosal, among other routes of administration in the present disclosure.

The words “administration” and “administering” in the present disclosure refer to the introduction of nicotinamide adenine dinucleotide, a drug, or a substance as a drug delivery method.

The term “administration” also refers to the methods of delivery of nicotinamide adenine dinucleotide molecular carriers of the invention, along with drugs, chemicals, nutraceuticals, or other substances by routes of administration that include topical, transdermal, dermal, intradermal, buccal, sublingual, intraoral, oral, nasal, intranasal, injection, intravenous, iontophoresis, or rectal methods.

The term “top-down” in the present disclosure refers to technology or methods of breaking down larger structures to generate sub-micron or nano-sized dimensional structure or nanostructured nicotinamide adenine dinucleotide or nano-drug delivery system for its use.

Top-down also refers to slicing or successive cutting of material to obtain nano-sized particles, nanostructures, and nanocomposites. It also refers to a step in a sequence of formulating or nano structuring nicotinamide adenine dinucleotide or nanostructured drug delivery system. Examples of top-down technology include mechanical milling, three rollers milling, jet milling, and sonication.

The term “bottom-up” in the present disclosure refers to technology or methods of forming single atoms and molecules into nanostructured nicotinamide adenine dinucleotide and nanosized drug delivery systems, nano-sized particles, nanostructures or nanocomposite carriers; and fabrication technologies forming lipid nanoparticles, polymer nanoparticles, lipid and polymer nanostructure and matrixes, hydrogels, nanogels, organogels, micelles, reverse micelles, premade and self-forming nano emulsions, microemulsions, liposomes ethosomes, nanospheres, nano-dimensional lipid compositions, and nano-dimension structures or matrixes.

Bottom-up refers to creating nanostructure product formations utilizing sedimentation and reduction techniques, sol-gel, green synthesis, spinning, and biochemical synthesis; with the utilization of processing techniques such ultrasonication, high-pressure homogenization, micro fluidization, high shear homogenization, and emulsion processes.

The terms “nano-structured delivery” or “nano-structure delivery” in the present disclosure refers to molecular structures and matrices of nicotinamide adenine dinucleotide, drugs, compounds, or chemicals in a nano-scale dimensional size range including nanoparticles, nano emulsions, lipid nanoparticles, polymer nanoparticles, lipid and polymer nanostructure and matrixes, hydrogels, nanogels, organogels, micelles, reverse micelles, premade and self-forming nano emulsions, microemulsions and ethosomes, nanospheres, or nanoparticle lipid compositions used to deliver NAD+, drugs, phytochemicals, compounds. and other agents.

The term “phospholipids” in the present disclosure refers to a tri-ester of glycerol with two fatty acids and one phosphate ion. Phospholipids include natural-occurring phospholipids such as phosphatidylcholine sphingosine, gangliosides, and phytosphingosine and combinations thereof derived from soy and lecithin that are preferable for use in this disclosure. They also include the synthetic phospholipids that include but are not limited to diacylglycerols, phosphatidic acids, phosphocholines, phosphoethanolamines, and phosphoglycerols.

The term “essential phospholipids” in the present disclosure refers to the highly purified extract of a specific fatty acid composition of phospholipids distinguished by their particularly high content of polyunsaturated fatty acids, predominantly linoleic acid (approximately 70%), linolenic acid, and oleic acid and with more than 75% of (3-sn-phosphatidyl) choline used to formulate the nanostructured nicotinamide adenine dinucleotide carrier composition. Besides phosphatidylcholine molecules, the essential phospholipid fraction includes phosphatidylethanolamine, phosphatidylinositol, and other lipids. Essential phospholipids include both hydrogenated and non-hydrogenated phospholipids manufactured from soy and sunflowers available from Lipoid and other suppliers.

The term “surfactant” in this disclosure refers to compounds that lower the surface tension (or interfacial tension) between two liquids or between a liquid and a solid, which act as emulsifiers, dispersants, wetting agents, and viscosity modifiers. In one embodiment, surfactants refer to amphiphilic molecules that are manufactured by chemical processes or are purified from natural sources or processes that can be anionic, cationic, nonionic, and zwitterionic.

The term “poloxamer”, also known as Pluronics® in this disclosure, refers to block copolymers of poly(ethylene oxide) and poly(propylene oxide), used in the synthesis of nanostructured carriers of nicotinamide adenine dinucleotide, or drug dosage forms, Poloxamers have an amphiphilic character and useful association and adsorption properties emanating from this use. Poloxamers are used in many applications that require solubilization or stabilization of compounds and have notable physiological properties.

The term “carbomer” in this disclosure refers to a polymer of acrylic acid cross-linked with a polyfunctional compound, hence, a poly (acrylic acid) or polyacrylate; a suspending agent for use in the synthesis of nanostructured carriers of nicotinamide adenine dinucleotide, or drug dosage forms.

The term “mucoadhesives” in the present disclosure refers to agents, molecules, chemicals, compounds that prolong the residence time of nanostructured carriers of nicotinamide adenine dinucleotide or drug, or substance at their sites of application that include the buccal and oral mucosa, skin and epidermal barriers, nasal mucosa, intestinal mucosa, vaginal mucosa, and rectum.

Examples of mucoadhesive agents include gums, mucin starches, chitosan, pectin, polymers, poloxamers, cellulose derivatives (methylcellulose, ethyl cellulose, hydroxyethyl cellulose, hydroxyl propyl cellulose, hydroxyl propyl methylcellulose, sodium carboxymethylcellulose, poly (acrylic acid) polymers (carbomers, polycarbophil), poly (hydroxyethyl methyl acrylate), poly (ethylene oxide), poly (vinyl pyrrolidone), poly (vinyl alcohol), (ethylene oxide), p (vinyl pyrrolidone), and poly (vinyl alcohol).

The term “ultrasonication” in this disclosure refers to methods in the assembly of nanostructured carriers or nanoparticles from ultrasound waves in which ultrasonic amplitudes generate intense cavitation by alternating high-pressure and low-pressure cycles. These disperse and break up particles down to the nanometer scale. In one embodiment, ultrasonication refers to high-power ultrasonic liquid processors also known as sonicators, ultrasonic homogenizers, sonochemical reactors, ultrasonic mixers, and ultrasonic wet-milling systems.

The term “milling” in this disclosure refers to methods in the assembly of nanostructured nicotinamide adenine dinucleotide, nanoparticles, and nanostructured compounds by which an external force is applied to a solid that leads to its break-up into smaller particles. In one embodiment milling refers to wet grinding carried out using methods as a roller ointment mill, tumbling ball mill, vibratory ball mill, a planetary ball mill, a centrifugal fluid mill, an agitating beads mill, a flow conduit beads mill, an annular gap beads mill, and wet jet mill. In one embodiment, milling refers to dry grinding by compression or by friction, using methods as a jet mill, a hammer mill, a shearing mill, a roller mill, a shock shearing mill, a ball mill, and a tumbling mill. In one embodiment milling refers to wet processes for preventing the condensation of the nanoparticles so formed and obtaining highly dispersed nanoparticles.

The term “homogenization” in this disclosure refers to methods of formulation of nano-structured nicotinamide adenine dinucleotide, drugs, chemicals of high-shear fluid processing reducing the size of droplets and particles in liquid-liquid dispersions to submicron sizes. In one embodiment, homogenization refers to high shear fluid processors such as the Ultra-Turrax, Kinematika Polytron, Ross, and Silverson processors used for particle reduction, and liquid mixing. In one embodiment, homogenization refers to high-pressure homogenization piston gap and micro fluidization methods such as APV Gaulin, Avestin, and Microfluidics homogenizers used for making nano-structures of NAD+ and nanoparticles.

The term “self-emulsifying drug delivery systems (SEDDS)” in this disclosure refers to lipid-based formulations that encompass isotropic mixtures of natural or synthetic oils, solid or liquid surfactants, and co-surfactants. When exposed to aqueous media of gastrointestinal fluids undergo self-emulsification to form oil-in-water nano emulsions or microemulsions. SEDDS are usually referred to as self-nanoemulsifying drug delivery systems (SNEDDS) or self-micro emulsifying drug delivery systems (SMEDDS) depending on the nature of the resulting dispersions formed following their dilution.

The term “nutraceutical” in the present disclosure refers to foods or food products, a product isolated or purified from foods. The term includes products that range from isolated nutrients, dietary supplements, vitamins, and phytonutrients minerals and herbal products,

The term “effective dose” in the present disclosure refers to a dose or concentration of a psychedelic drug or medicinal mushroom or a nutraceutical or petrochemical or natural compound or bioactive substance that produces a biological response.

The term “bioactive substance” in the present disclosure refers to a substance or compound or molecules having an effect on, or causing a reaction, or triggers a response in living tissue or on a living organism, presenting therapeutic potential.

Disclosed are the compositions and methods of a nanostructured nicotinamide adenine dinucleotide drug delivery system comprised of two formulations: a nanostructured nicotinamide adenine dinucleotide composition and chemical drug delivery enhancer composition and co-administering them by one of the many drug delivery routes including topical, transdermal, intraoral, sublingual, buccal, intranasal, oral, ocular, rectal, injection, or iontophoretic patch administration.

Disclosed are the compositions and methods of a nicotinamide adenine dinucleotide drug delivery system comprised of two formulations: a nicotinamide adenine dinucleotide dosage form and a chemical drug delivery enhancer and co-administering them by one of the many drug delivery routes including topical, transdermal, intraoral, sublingual, buccal, intranasal, oral, ocular, rectal, injection, or iontophoretic patch administration.

Disclosed are the compositions and methods of a drug delivery system comprised of two formulations: a pharmaceutical, nutraceutical, cosmeceutical, cannabinoid, phytochemical, psychedelic drug, or chemical composition and chemical delivery enhancer composition; and co-administering them by different routes and methods of drug delivery that includes topical, or transdermal, or intraoral, or sublingual, or buccal, or intranasal, or oral, or ocular, or rectal, or injection, or iontophoretic patch administration.

Disclosed are the compositions and methods for a nanostructured nicotinamide adenine dinucleotide drug delivery system co-administering dosage form of two formulations: a dosage form of a nanostructured nicotinamide adenine dinucleotide composition and a dosage form of chemical delivery enhancer composition as a carrier vehicle of nicotinamide adenine dinucleotide into the body.

One embodiment of this disclosure teaches the compositions, methods of usage, and formulations of nanostructured bioidentical nicotinamide adenine dinucleotide (BioNAD+) dosage form composition synthesized from bioidentical nicotinamide adenine dinucleotide and co-administering it with a dosage form of chemical drug delivery enhancer composition.

One embodiment of this disclosure teaches compositions, methods, and formulations of nanostructured nicotinamide adenine dinucleotide compositional dosage forms synthesized from lyophilized, or non-lyophilized nicotinamide adenine dinucleotide, and co-administering it with a chemical drug delivery enhancer composition.

One embodiment of this disclosure teaches compositions, methods, and formulations of nicotinamide adenine dinucleotide derivatives, or analogs, or precursors that include NR, or NMN, or NADH, or NADP, or NADPH, and co-administered them with a chemical drug delivery enhancer composition.

This disclosure teaches methods of co-administering dosage forms of a nanostructured nicotinamide adenine dinucleotide composition with a chemical delivery enhancer composition dosage form optimized for effective delivery into the body different routes and methods of drug delivery that includes topical, or transdermal, or intraoral, or sublingual, or buccal, or intranasal, or oral, or ocular, or rectal, or injection, or iontophoretic patch administration.

This disclosure teaches compositions and methods of formulating a nanostructured nicotinamide adenine dinucleotide drug delivery system for delivering stabile nicotinamide adenine dinucleotide dosage forms into the body for increasing nicotinamide adenine dinucleotide dosage delivery concentrations, or increasing nicotinamide adenine dinucleotide bioavailability, or improving nicotinamide adenine dinucleotide pharmacokinetics.

This disclosure teaches compositions and methods of nicotinamide adenine dinucleotide delivery for overcoming nicotinamide adenine dinucleotide problems of formulation instability, or low solubility, or low bioavailability from the intestinal tract after oral administration of nicotinamide adenine dinucleotide from drug delivery vehicles.

Numerous chemical delivery enhancers for nicotinamide adenine dinucleotide can degrade nicotinamide adenine dinucleotide molecules, drug delivery nanostructures, drug carriers, or drug delivery vehicles. Some chemical delivery enhancer ingredients for nicotinamide adenine dinucleotide are insoluble in nicotinamide adenine dinucleotide. Their use in a formulation of nicotinamide adenine dinucleotide can produce unstable nicotinamide adenine dinucleotide dosage forms that quickly degrade and are unsuitable for use.

This invention teaches the co-administration of a separate chemical drug delivery enhancer dosage form in conjunction with a nicotinamide adenine dinucleotide dosage form for improving nicotinamide adenine dinucleotide delivery into the body by different routes without destabilizing or degrading nicotinamide adenine dinucleotide or compromising the integrity of the nicotinamide adenine dinucleotide drug delivery system, nanostructures, or drug delivery vehicle.

This disclosure teaches compositions and methods of formulating a nanostructured nicotinamide adenine dinucleotide drug delivery system producing synergistic effects between molecules of a chemical drug delivery enhancer composition and molecules of a nicotinamide adenine dinucleotide composition to augment nicotinamide adenine dinucleotide levels in the body and for therapeutic applications.

In one embodiment, this disclosure teaches additive or synergistic effects between two or more molecules of chemical delivery enhancer agents that are greater than a single chemical agent at improving the delivery of nicotinamide adenine dinucleotide, nucleotides, or nicotinamide adenine dinucleotide analogs.

In one embodiment, this disclosure teaches additive or synergistic effects between two or more molecules of chemical delivery enhancer agents that are greater than a single chemical agent at improving the delivery of different classes of compounds that encompass drugs, nutraceuticals, cosmeceuticals, cannabinoids, psychedelic drugs, bioactive compounds, chemicals, and other agents.

This disclosure teaches compositions and methods of formulating and manufacturing dosage forms of a chemical delivery enhancer product and a nanostructured nicotinamide adenine dinucleotide product dosage form in a sequential or unified process of production.

This disclosure teaches composition and methods of formulating and co-administering dosage forms of two nanostructured nicotinamide adenine dinucleotide drug delivery system formulations, a nanostructured nicotinamide adenine dinucleotide composition, and a synergistic chemical drug delivery enhancer composition, for improving the pharmacokinetics of nicotinamide adenine dinucleotide that include area under the curve (AUC) of plasma concentration-time profile, maximum plasma concentration (Cₘₐₓ), time to maximum plasma concentration (Tₘₐₓ) and volume of distribution (V_{d} or Vₛₛ), elimination half-life (t½), and clearance (CL).

This disclosure teaches compositions and methods of a nanostructured nicotinamide adenine dinucleotide drug delivery system for boosting endogenous nicotinamide adenine dinucleotide levels and systemic concentrations of nicotinamide adenine dinucleotide.

This disclosure teaches a nanostructured nicotinamide adenine dinucleotide drug delivery system as a non-invasive alternative to nicotinamide adenine dinucleotide administration by injection.

At present, intravenous infusion of nicotinamide adenine dinucleotide is the only recognized effective means of clinically increasing systemic nicotinamide adenine dinucleotide levels. The nicotinamide adenine dinucleotide precursors including nicotinic acid (NA), nicotinamide (Nam), nicotinamide riboside (NR), nicotinamide mononucleotide (NMN), nicotinic acid riboside (NAR) is fractionally effective, slow-acting, and rate-limited at increasing measurable changes in nicotinamide adenine dinucleotide levels.

This disclosure teaches compositions and methods of a nanostructured nicotinamide adenine dinucleotide drug delivery system for delivering nicotinamide adenine dinucleotide into the skin, and preventing or treating skin aging, or skin disorders.

This disclosure teaches compositions, methods, and manufacturing a nanostructured nicotinamide adenine dinucleotide drug delivery system administering a nicotinamide adenine dinucleotide dosage form composition into the nasal cavity for delivering nicotinamide adenine dinucleotide into the bloodstream.

One embodiment of this invention teaches compositions, methods and manufacturing a nanostructured nicotinamide adenine dinucleotide drug delivery system administering a nicotinamide adenine dinucleotide dosage form composition into the nasal cavity and delivering nicotinamide adenine dinucleotide directly into the brain along the olfactory nerve or trigeminal nerve.

One embodiment of this invention teaches compositions, methods and manufacturing a nanostructured nicotinamide adenine dinucleotide drug delivery system, administering a nicotinamide adenine dinucleotide dosage form, nicotinamide adenine dinucleotide dosage form composition to the sublingual or buccal mucosa for delivering nicotinamide adenine dinucleotide into the bloodstream.

One embodiment of this invention teaches compositions, methods, and manufacturing a nanostructured nicotinamide adenine dinucleotide drug delivery system, administering a nicotinamide adenine dinucleotide dosage for, nicotinamide adenine dinucleotide dosage form composition orally to the gastrointestinal tract for delivering nicotinamide adenine dinucleotide to the bloodstream.

One embodiment of this invention teaches compositions, methods, and manufacturing a nanostructured nicotinamide adenine dinucleotide drug delivery system, administering the nicotinamide adenine dinucleotide dosage form composition for transmucosal vaginal delivery of nicotinamide adenine dinucleotide for delivering nicotinamide adenine dinucleotide to the bloodstream.

One embodiment of this invention teaches compositions, methods, and manufacturing a nanostructured nicotinamide adenine dinucleotide drug delivery system, administering a nicotinamide adenine dinucleotide dosage form composition for ocular delivery of nicotinamide adenine dinucleotide to the bloodstream.

One embodiment of this invention teaches compositions, methods, and manufacturing a nanostructured nicotinamide adenine dinucleotide drug delivery system, administering a nicotinamide adenine dinucleotide dosage form composition for rectal delivery of nicotinamide adenine dinucleotide to the bloodstream.

This disclosure teaches compositions and methods of formulating and manufacturing a dosage form of nanostructured nicotinamide adenine dinucleotide and co-administering it with a dosage form of chemical drug delivery enhancer.

This disclosure teaches compositions and methods of formulating and manufacturing a nanostructured nicotinamide adenine dinucleotide dosage form composition by a top-down or bottom-up process of assembly.

One embodiment of this disclosure teaches compositions and methods of formulating and manufacturing a nanostructured nicotinamide adenine dinucleotide dosage form combining one or more top-down processes of assembly.

One embodiment of this disclosure teaches compositions and methods of formulating and manufacturing a nanostructured nicotinamide adenine dinucleotide dosage form combining one more bottom-up process of assembly.

One embodiment of this disclosure teaches compositions and methods of formulating and manufacturing a nanostructured nicotinamide adenine dinucleotide dosage form composition by processes that include homogenization techniques, or hot high-pressure homogenization technique, or cold high-pressure homogenization technique, or melt emulsification, or ultrasound (ultrasonication), or high shear homogenization, or micro fluidization, or microemulsion technique, or emulsification-solvent evaporation technique, or solvent displacement, or injection technique, or emulsification-solvent diffusion technique, or phase inversion technique, or film ultrasonication dispersion technique, or multiple emulsion techniques, or other manufacturing processes.

This disclosure teaches a preferred method of synthesizing nanostructures of nicotinamide adenine dinucleotide combining ultra-sonication, high shear homogenization, and milling techniques methods of production.

One embodiment of this disclosure teaches compositions and methods of formulating and manufacturing a nanostructured nicotinamide adenine dinucleotide dosage form, combining microfluidation and emulsification or milling techniques methods of production.

One embodiment of this disclosure teaches compositions and methods of formulating and manufacturing a nanostructured nicotinamide adenine dinucleotide dosage form, combining high-pressure homogenization or microfludization as the method of assembly.

One embodiment of this disclosure teaches compositions and methods of formulating and manufacturing a nanostructured nicotinamide adenine dinucleotide dosage by a combination of three techniques sequentially performed for dispersion comprising milling (physical grinding), or homogenization (high-speed stirring emulsification), or ultrasonic processing (high wattage flow through ultrasound sonification).

This disclosure teaches of compositions and methods of a “nicotinamide adenine dinucleotide base formulation”, comprised of the synergistic ingredients for systemic delivery of nicotinamide adenine dinucleotide through different routes of drug delivery that include topical, or transdermal, or sublingual, or buccal, or intranasal, oral, or ocular, or rectal, or injection or iontophoretic patch methods of administration.

This disclosure teaches compositions and methods of a nanostructured nicotinamide adenine dinucleotide delivery system, co-administering a nanostructured nicotinamide adenine dinucleotide base formulation with a chemical delivery enhancer formulation of synergistic ingredients for delivering nicotinamide adenine dinucleotide into the systemic circulation by different drug delivery pathways, and improving the pharmacokinetics and therapeutic effectiveness of nicotinamide adenine dinucleotide.

### EXAMPLE 1

### Nanostructured Nicotinamide Adenine Dinucleotide Drug Delivery” Carrier Base Formulation:

Nicotinamide adenine dinucleotide (lyophilized or non-lyophilized): 5%-25%,

Essential Phospholipids: 5%-30%,

Lipids and Fatty Acids: 0%-30%,

Water: 0%-20%

Glycols: 0%-75%

Azone: 0%-10%

Alcohols: 0%-50%

Surfactants: 2%-10%

Mucoadhesive: 0%-5%

Non-phosphate buffer: 2%-8%

Polymers and Macromolecules: 0%-25%

One embodiment of this disclosure teaches compositions and methods of formulating a nicotinamide adenine dinucleotide carrier base formulation with ingredients producing stable nanostructured nicotinamide adenine dinucleotide dosage forms for delivering nicotinamide adenine dinucleotide across the stratum corneum and dermis into the bloodstream, and augmenting nicotinamide adenine dinucleotide levels.

One embodiment of this disclosure teaches compositions and methods of formulating an nicotinamide adenine dinucleotide carrier base formulation with ingredients for producing a stabile nanostructured nicotinamide adenine dinucleotide dosage form for protecting nicotinamide adenine dinucleotide from hydrolysis and degradation in the intestinal after oral administration, and delivering nicotinamide adenine dinucleotide across the intestinal lumen into the bloodstream for therapeutic applications, and augmenting nicotinamide adenine dinucleotide levels.

One embodiment of this disclosure teaches compositions and methods of a nanostructured nicotinamide adenine dinucleotide drug delivery system consisting of nanostructured nicotinamide adenine dinucleotide base carrier formulation co-administered with a drug delivery enhancer formulation for delivering nicotinamide adenine dinucleotide across the stratum corneum into the bloodstream.

One embodiment of this disclosure teaches compositions and methods of formulating a nanostructured nicotinamide adenine dinucleotide drug delivery system, co-administering a nanostructured nicotinamide adenine dinucleotide carrier base formulation with a drug delivery enhancer formulation of synergistic ingredients for protecting nicotinamide adenine dinucleotide from pre-systemic metabolism, or hydrolysis or degradation in the intestinal tract.

One embodiment of this disclosure teaches compositions and methods of a nanostructured nicotinamide adenine dinucleotide drug delivery system, co-administering a nanostructured nicotinamide adenine dinucleotide base formulation with a synergistic chemical drug delivery enhancer formulation, and delivering nicotinamide adenine dinucleotide across the stratum corneum into the bloodstream for therapeutic applications, or improving nicotinamide adenine dinucleotide pharmacokinetics.

Preferred phospholipids for synthesizing nanostructured carries vehicles of nicotinamide adenine dinucleotide of this disclosure include Lipoid Phospholipon 90G, Lipoid Phospholipon 90 H, Lipoid Phospholipon 85 G, Lipoid S 75, Lipoid S 40, Lipoid S 80, Lipoid E80, Lipoid Phosal 75 SA, Lipoid Phosal 53 MCT, Alcolec 40 P, Lipoid S 45, Lipoid S 75, Lipoid H 50, Phosal 40 IP, Phosal 35 SB, Lipoid H 90, Lipoid H 100, and other pharmaceutical phospholipids

Preferred synergistic additive compounds for synthesizing nanostructures and drug carrier vehicles of nicotinamide adenine dinucleotide of this disclosure include: polysorbates (tweens), sorbitan monooleate, polyoxyethylene sorbitan monooleate, lecithins, and laureth-3, or isopropyl palmitate.

Preferred polymers for synthesizing nanostructures and drug carrier vehicles of nicotinamide adenine dinucleotide of this disclosure include poloxamer, pectin, carbomer, cellulose, hydroxypropyl methylcellulose, chitosan, hyaluronic acid, dimethicone, coco-caprylate/caprate, silicone elastomers, cyclopentosiloxane gel, alginate, or maltodextrin.

Preferred lipids and fatty acids for synthesizing nanostructure carrier vehicles of nicotinamide adenine dinucleotide in this disclosure include: fatty acids, triglycerides triacylglycerols, acylglycerols, fats, waxes, cholesterol, sphingolipids, glycerides, sterides, cerides, glycolipids, sulfolipids, lipoproteins, stearic acid, palmitic acid, myrisitic acid, oleic acid, sunflower oil, soy oil, caprylic acid, hempseed oil, palmitoleic acid, linoleic acid, decanoic acid, lauric acid, decanoic acid, undecanoic acid, stearic acid, free fatty acid alcohols, stearyl alcohol, cetyl alcohol, myristyl alcohol, lauryl alcohol, triglycerides, trimyristin, tripalmitin, trilaurin, bees wax, cetyl palmitate, carnuba wax, cannabis wax extract, mono, di and triglycerides mixtures, Suppocire NC, witepsol bases, glyceryl monostearate, glyceryl behenate, palmitostearate, softisan, cacao butter, castor oil, anhydrous milk fat, or hydrogenated palm oil.

Preferred non-phosphate buffers for synthesizing nanostructure carrier vehicles of nicotinamide adenine dinucleotide in this disclosure include TRIS, Trizma, MES buffers, glycine citrate, limonene, borate, and carbonate.

### Methods and Compositions of Stabilized Nicotinamide Adenine Dinucleotide

An effective nicotinamide adenine dinucleotide dosage form maintains physical, chemical, therapeutic, microbiological, and toxicological stability within its stated shelf-life. Unfortunately, nicotinamide adenine dinucleotide is susceptible to chemical, physical, and microbiological degradation. Degradation is more problematic for liquid forms of nicotinamide adenine dinucleotide. They can degrade over time after manufacturing, during storage, and before use. This is critical because drug degradation often leads to loss of potency and therapeutic effects.

Nicotinamide adenine dinucleotide degradation can result from chemical degradation pathways that include hydrolysis/solvolysis, oxidation, photolysis, polymerization, dehydration, isomerization, racemization, or chemical incompatibilities. physical degradation pathways that include polymorphism, particle size, vaporization, evaporation, temperature, efflorescence, hygroscopy, or deliquescence, or from microbial degradation pathways.

Water alone should not be used to prepare formulations and fluid compositions solutions of nicotinamide adenine dinucleotide since it tends to be acidic and would decompose nicotinamide adenine dinucleotide. Phosphate buffers accelerate the destruction of nicotinamide adenine dinucleotide

This disclosure teaches compositions and methods of stable dosage forms of nicotinamide adenine dinucleotide that resist degradation.

This disclosure teaches compositions and methods for stable dosage forms of nanostructured nicotinamide-adenine dinucleotides of nicotinamide adenine dinucleotide (oxidized nicotinamide-adenine dinucleotide), NADH (reduced nictonamide-adenine dinucleotide), NADP+ (oxidized nicotinamide-adenine dinucleotide phosphate, NADPH (reduced nicotinamide-adenine dinucleotide phosphate) and nicotinamide-adenine dinucleotide derivatives, or analogs.

This disclosure teaches compositions and methods of stable dosage forms of nanostructured nicotinamide adenine dinucleotide formulated with lyophilized nicotinamide adenine dinucleotide or non-lyophilized nicotinamide adenine dinucleotide molecular compositions

This disclosure teaches compositions and methods of manufacturing stable dosage forms of nicotinamide adenine dinucleotide, or nanostructured nicotinamide adenine dinucleotide by incorporating solvents in a drug delivery carrier composition that include glycols for inhibiting nicotinamide adenine dinucleotide degradation.

This disclosure teaches compositions and methods of formulating stable dosage forms of nicotinamide adenine dinucleotide, or nanostructured nicotinamide adenine dinucleotide by incorporating phospholipids in a drug delivery carrier composition for inhibiting nicotinamide adenine dinucleotide degradation.

This disclosure teaches compositions and methods of formulating stable dosage forms of nicotinamide adenine dinucleotide or nanostructured nicotinamide adenine dinucleotide by incorporating polymers in a drug delivery carrier composition for inhibiting nicotinamide adenine dinucleotide degradation.

This disclosure teaches compositions and methods of formulating stable dosage forms of nicotinamide adenine dinucleotide or nanostructured nicotinamide adenine dinucleotide by incorporating non-phosphate buffers in a drug delivery carrier composition for inhibiting nicotinamide adenine dinucleotide degradation.

This disclosure teaches compositions and methods of formulating stable dosage forms nicotinamide adenine dinucleotide or nanostructured nicotinamide adenine dinucleotide for therapy with a stabilizer or protective molecular structure for preventing physiochemical degradation of nicotinamide adenine dinucleotide.

In one embodiment of this disclosure, the protective molecular structure is a Nano dimensional structure.

In one embodiment of this disclosure, the protective molecular structure is a lipid or polymer nanodimensional structure.

In one embodiment of this disclosure, the protective molecular structure is a lipid or phospholipid.

In one embodiment of this disclosure, the protective molecular structure is a polymer.

In one embodiment of this disclosure, the protective molecular structure is an emulsion, or nanoemulsion, or microemulsion, or self-forming nanoemulsion, or self-forming microemulsion.

In one embodiment of this disclosure, the protective molecular structure is a film system.

In one embodiment of this disclosure, the protective molecular structure is a microparticle.

In one embodiment of this disclosure, the protective molecular structure is a dendrimer system.

In one embodiment of this disclosure, the stabilizer is a polymer or macromolecule, or shellac applied to particles or tablets of nicotinamide adenine dinucleotide.

In one embodiment of this disclosure, the stabilizer is a lipid.

In one embodiment of this disclosure, the stabilizer is a phospholipid.

In one embodiment of this disclosure, the stabilizer is a polymer.

In one embodiment of this disclosure, the stabilizer is a spin trap compound.

In one embodiment of this disclosure, the stabilizer is EDTA.

In one embodiment of this disclosure, the stabilizer is a non-phosphate buffer.

In one embodiment of this disclosure, the stabilizer is a nanoparticle structure.

In one embodiment of this disclosure, the stabilizer is a hydrogel.

In one embodiment of this disclosure, the stabilizer is an organogel.

In one embodiment of this disclosure, the stabilizer is a micelle.

In one embodiment of this disclosure, the stabilizer is a reverse micelle.

In one embodiment of this disclosure, the stabilizer is nanoparticle lipid composition.

In one embodiment of this disclosure, the stabilizer is an emulsion

Compositions and Methods of a Drug Delivery Enhancer for Nicotinamide Adenine Dinucleotide

This disclosure teaches compositions and methods of formulating and manufacturing a dosage form of chemical drug delivery enhancer formulation and co-administering it with a dosage form of a nanostructured nicotinamide adenine dinucleotide formulation, that comprise a nanostructured nicotinamide adenine dinucleotide drug delivery system.

This disclosure teaches chemical drug delivery enhancers and physical methods for drug delivery enhancement of nicotinamide adenine dinucleotide for improving the delivery of nicotinamide adenine dinucleotide into the body by different routes and methods of drug delivery that include topical, transdermal, sublingual, buccal intraoral, intranasal, oral, ocular, rectal, injection, or iontophoretic patch methods of administration.

In one embodiment, this disclosure teaches chemical delivery enhancers or physical method delivery enhancers of nicotinamide adenine dinucleotide that improve the delivery of nicotinamide adenine dinucleotide through the skin for entry into the systemic circulation.

In one embodiment, this disclosure teaches chemical delivery enhancers of nicotinamide adenine dinucleotide that improve delivery or transport molecules of nicotinamide adenine dinucleotide through gastrointestinal lumen membranes for entry into the systemic circulation.

In one embodiment, this disclosure teaches chemical delivery enhancers of nicotinamide adenine dinucleotide that improve delivery or transport molecules of nicotinamide adenine dinucleotide through nasal membranes into the systemic circulation, or along the trigeminal or olfactory nerves in the nasal cavity for entry into the brain.

In one embodiment, this disclosure teaches chemical delivery enhancers of nicotinamide adenine dinucleotide that improve delivery or transport molecules of nicotinamide adenine dinucleotide through sublingual or buccal oral mucosa for entry into the systemic circulation.

In one embodiment, this disclosure teaches compositions and methods of chemical delivery drug enhancer dosage forms with synergistic effects for improving the delivery of dosage forms of different compounds and chemicals into the body from their co-administration that include pharmaceuticals, nutraceuticals, cosmeceuticals, hormones, cells, genomic materials, enzymes, botanical extracts, peptides, molecular structures, growth hormones, viruses, bacteria, toxins, neurotoxins, bioactive agents, dietary supplements, and other chemicals.

In one embodiment, this disclosure teaches compositions and methods of chemical drug delivery enhancer dosage forms with synergistic effects for improving the delivery of dosage forms of different compounds and chemicals by different routes of drug delivery and include pharmaceuticals, nutraceuticals, cosmeceuticals, hormones, cells, genomic materials, enzymes, botanical extracts, peptides, molecular structures, growth hormones, viruses, bacteria, toxins, neurotoxins, bioactive agents, dietary supplements, and other chemicals.

In one embodiment, this disclosure teaches chemical drug delivery enhancer compositions for co-administration with resveratrol or pterostilbene, or oleic acid other compounds to maintain nicotinamide adenine dinucleotide concentrations in the body or inhibit the breakdown and consumption of nicotinamide adenine dinucleotide.

In one embodiment, this disclosure teaches chemical drug delivery enhancer compositions for co-administration with a drug, or chemical, or botanical extract, or cannabinoid, phytochemical or nutraceutical, or genomic material, or other compounds, or agents dosage form to improve their delivery into the body, or increase their concentration in the body, or improve their pharmacokinetics, or improve their efficacy.

In one embodiment, this disclosure teaches chemical delivery enhancer compositions optimized for co-administration with a drug, or chemical, or botanical extract, or cannabinoid, or phytochemical or nutraceutical, or genomic material, or other compounds, or agent dosage for improving their delivery to the body by different routes and methods of administration.

In one embodiment, this disclosure teaches chemical drug delivery enhancer dosage forms optimized for co-administration with a cannabinoid dosage form for delivering cannabinoids to the body by different routes and methods of cannabis delivery.

In one embodiment, this disclosure teaches chemical delivery enhancer dosage forms optimized for co-administration with psychedelic drugs for improving their delivery into the body by different routes and methods of administration.

This disclosure teaches compositions and methods of chemical drug delivery enhancers for increasing the rate and/or extent to which co-administered nicotinamide adenine dinucleotide reach the systemic circulation unchanged or with increased bioavailability.

This disclosure teaches compositions and methods of formulating a chemical delivery enhancer dosage form containing following ingredients for co-administration with nicotinamide adenine dinucleotide, or pharmaceuticals, or nutraceuticals, or cosmeceuticals, or cannabinoids, hormones, or cells, or genomic materials, or enzymes, or botanical extracts, peptides, molecular structures, growth hormones, viruses, bacteria, toxins, vaccines, or neurotoxins, or bioactive agents, or dietary supplements, or chemicals. The chemical delivery enhancer can be sulphoxides (such as dimethylsulphoxide, DMSO); polysorbates (polysorbate 20, polysorbate 60, polysorbate 80); azones (laurocapram); pyrrolidones (2-pyrrolidone, 1-dodecyl-2-pyrrolidinone, N-methyl-2-pyrrolidone, 2P); alcohols and alkanols (ethanol, or decanol); surfactants (anionic, cationic, zwitterionic, nonionic surfactants 5-hydroxystearate); glycerols; glycols (propylene glycol, triethylene glycol); polyglycols; fatty acids, (oleic acid, palmitoleic acid, linoleic acids, decanoic acid, lauric acid, decanoic acid, undecanoic acid, stearic acid); acidifiers (citric acid, malic acid); lipids; phospholipids; terpenes & terpenoids (menthol, d-limonene, 1,8 cineol); esters (isosorbide dimethyl ether, Isopropyl myristate, isopropyl palmitate); cyclic oligosaccharides (cyclodextrins, methyl-β-cyclodextrin), vesicles and nanostructures (emulsomes, liposomes, nanocasules, lipid nanoparticles, and polymer nanoparticles); or other compounds (lactose esters, sodium dilauramidoglutamide lysine, ammonium mysristoyl chitosan); or a combination thereof.

In one embodiment, this disclosure teaches methods of chemical delivery enhancer dosage forms for co-administration with nicotinamide adenine dinucleotide dosage forms that contain bioavailability enhancers for increasing nicotinamide adenine dinucleotide dosage delivery across the cell membrane, or increasing nicotinamide adenine dinucleotide bioavailability, or potentiating the nicotinamide adenine dinucleotide molecular conformational interaction, acting as receptors for nicotinamide adenine dinucleotide molecules, and making target cells more receptive to them.

This invention teaches the physical delivery enhancers for nicotinamide adenine dinucleotide dosage forms that include iontophoresis, electroporation, sonophoresis, use of laser radiation and radiofrequency, microneedles, or thermal ablation.

This disclosure teaches of compositions and methods of formulating a chemical delivery enhancer base formula of synergistic ingredients for co-administration with a dosage form of nicotinamide adenine dinucleotide, or pharmaceuticals, or nutraceuticals, or cosmeceuticals, or cannabinoids, hormones, or cells, or genomic materials, or enzymes, or botanical extracts, peptides, molecular structures, growth hormones, viruses, bacteria, toxins, vaccines, or neurotoxins, or bioactive agents, or dietary supplements, or chemicals.

### EXAMPLE 2

### Chemical Drug Delivery Enhancer Base Formulation

Alcohols (ethanol): 10%-75%

Glycols (propylene glycol) 10%-90%

Azone (Laurocapram 1-dodecylazacycloheptan-2-one): 2%-12%

Fatty acids (cis-oleic acid): 2%-12%

Surfactants (polysorbate 20, polysorbate 60): 1%-12%

Pyrrolidone (2-pyrrolidone): 1.5% -8%

Ethers (isosorbide dimethyl ether): 2%-10%

Terpenes & Essential Oils (d-limonene, 1,8 cineol, I-menthol): 2%-15%.

This disclosure teaches compositions and methods of formulating chemical drug delivery enhancers as a topical cream or gel, or adhesive skin patch, or another vehicle, and for administering prolonged or controlled delivery of nicotinamide adenine dinucleotide.

One embodiment of this disclosure teaches compositions and methods of formulating chemical drug delivery enhancers for administering prolonged or controlled delivery of pharmaceuticals, nutraceuticals, cosmeceuticals, cannabinoids, psychedelic drugs, hormone, cells, enzymes, botanical extracts, peptides, molecular structures, growth hormones, viruses, bacteria, toxins, vaccines, or neurotoxins, bioactive agents, dietary supplements, or chemicals.

One embodiment of this disclosure teaches methods and compositions of formulating a nicotinamide adenine dinucleotide drug delivery system that co-administers nicotinamide adenine dinucleotide with a chemical delivery that are combined into a single product dosage form before packaging or administration.

This disclosure teaches methods and compositions of a nanostructured drug delivery system containing pharmaceuticals, nutraceuticals, cosmeceuticals, cannabinoids, psychedelic drugs, hormones, cells, enzymes, botanical extracts, peptides, molecular structures, growth hormones, viruses, bacteria, toxins, vaccines, neurotoxins, bioactive agents, dietary supplements, or chemicals and co-administering a chemical drug delivery enhancer that is combined into a single product dosage form before packaging or administration.

One embodiment of this disclosure teaches compositions and methods of formulating chemical drug delivery enhancers for co-administration with inhibitor molecules of CD38 and PARPs for increasing nicotinamide adenine dinucleotide levels.

This disclosure teaches compositions and methods for formulating chemical drug delivery enhancers for co-administration with apigenin, quercetin, resveratrol, or pterostilbene, and other flavonoids for increasing nicotinamide adenine dinucleotide levels.

Compositions and Methods of Nanostructured Nicotinamide Adenine Dinucleotide Therapy

This disclosure teaches compositions and methods of co-administering nanostructured a nicotinamide adenine dinucleotide drug dosage form with a chemical drug delivery enhancer dosage form for administering nicotinamide adenine dinucleotide therapy.

This disclosure teaches compositions and methods of formulating and administering a nanostructured nicotinamide adenine dinucleotide drug delivery system for preventing, healing, or treating a wide range of medical diseases, pathologies, and metabolic abnormalities that are related to low levels of nicotinamide adenine dinucleotide.

This disclosure teaches compositions and methods of formulating and delivering nanostructured nicotinamide adenine dinucleotide to the body for treating conditions related to the depletion of nicotinamide adenine dinucleotide that include: decreased nicotinamide adenine dinucleotide biosynthesis, excessive nicotinamide adenine dinucleotide consumption, excessive DNA damage from free radical or ultraviolet attack, poly(ADP-ribose) polymerase (PARP) activation, chronic immune activation, inflammatory cytokine production, or accelerated CD38 activity decline in nicotinamide adenine dinucleotide levels.

This disclosure teaches compositions and methods of a nanostructured bioidentical nicotinamide adenine dinucleotide dosage form for nicotinamide adenine dinucleotide replacement therapy.

In one embodiment, this disclosure teaches composition and methods of a nanostructured nicotinamide adenine dinucleotide drug delivery system to prevent or treat suboptimal nicotinamide adenine dinucleotide levels that can contribute to aging disorders or metabolic conditions that include DNA damage, epigenetic alteration, deregulated nutrient-sensing, loss of proteostasis, altered cellular communication, cellular senescence, stem cell exhaustion, mitochondrial dysfunction, compromised autophagy, or telomere attrition.

In one embodiment, this disclosure teaches compositions and methods of a nanostructured nicotinamide adenine dinucleotide drug delivery system for treating metabolic conditions related to low nicotinamide adenine dinucleotide levels, or that respond to nicotinamide adenine dinucleotide replacement therapy or exogenous nicotinamide adenine dinucleotide. These include DNA damage, epigenetic alteration, deregulated nutrient-sensing, loss of proteostasis, altered cellular communication, cellular senescence, stem cell exhaustion, mitochondrial dysfunction, compromised autophagy, or telomere attrition.

In one embodiment, this disclosure teaches compositions and methods of improving the NAD+ metabolome, treating degenerative conditions, or treating aging disorders with a nanostructured nicotinamide adenine dinucleotide drug delivery system.

This disclosure teaches compositions and methods of a nanostructured nicotinamide adenine dinucleotide drug delivery system for increasing mitochondrial ATP generation or bioenergetics.

This disclosure teaches compositions and methods of a nanostructured nicotinamide adenine dinucleotide drug delivery system for improving or preventing a decline in athletic, ergogenic cardiovascular, or muscular performance.

In one embodiment, this disclosure teaches compositions and methods of a nanostructured nicotinamide adenine dinucleotide drug delivery system for improving cardiovascular or respiratory functions.

In one embodiment, this disclosure teaches compositions and methods of a nanostructured nicotinamide adenine dinucleotide drug delivery system administered nicotinamide adenine dinucleotide to the skin for treating or healing muscle injury of damage below the area of application.

This disclosure teaches compositions and methods of a nanostructured nicotinamide adenine dinucleotide drug delivery system for treating or preventing cognitive decline or neurological disorders.

This disclosure teaches compositions and method of a nanostructured nicotinamide adenine dinucleotide drug delivery system for treating, or preventing substance abuse, or addictive behavior.

This disclosure teaches compositions and methods of a nanostructured nicotinamide adenine dinucleotide delivery system for protecting neurons from traumatic injury, ischemia, or stroke.

This disclosure teaches compositions and methods of a nanostructured nicotinamide adenine dinucleotide drug delivery system for treating or preventing neurodegenerative conditions that include Alzheimer's, Parkinson's, Huntington's diseases, or amyotrophic lateral sclerosis.

### Methods and Compositions of Nicotinamide Adenine Dinucleotide Transdermal Drug Delivery

This disclosure teaches compositions and methods for a transdermal nanostructured nicotinamide adenine dinucleotide drug delivery system comprised of two formulations: a nanostructured nicotinamide adenine dinucleotide composition and a chemical drug delivery enhancer, and co-administering them through the skin's protective stratum corneum barrier intact by “transcellular absorption” and passes through the deeper epidermis to the dermal layer for systemic absorption via the dermal microcirculation.

One embodiment of this disclosure teaches compositions and methods of a transdermal drug delivery system comprised of two formulations: a pharmaceutical, or nutraceutical, or cosmeceutical, or cannabinoid, or phytochemical, or tryptamine, or phenethylamine, or ergoline, or medicinal mushroom, or psychedelic drug, or chemical drug delivery carrier vehicle and a chemical drug delivery enhancer dosage form composition, and co-administering them through the skin's protective stratum corneum barrier whereas the cargo passes through the deeper epidermis to the dermal layer for systemic absorption via the dermal microcirculation.

This disclosure teaches compositions methods of transdermal delivery of nanostructured nicotinamide adenine dinucleotide as a non-invasive alternative to parenteral routes of nicotinamide adenine dinucleotide administration. For circumventing issues such as needle phobia while providing a large surface area of skin for administration.

This disclosure teaches compositions methods of transdermal delivery of a nanostructured nicotinamide adenine dinucleotide drug dosage form for increasing nicotinamide adenine dinucleotide bioavailability and improving the pharmacokinetics of nicotinamide adenine dinucleotide.

One embodiment of this disclosure teaches compositions and methods of intradermal delivery of a nanostructured nicotinamide adenine dinucleotide drug dosage composition across the stratum corneum to epidermal and dermal layers as the target of nicotinamide adenine dinucleotide delivery.

### EXAMPLE 3

### Formulation of a Nanostructured Bioidentical Nicotinamide Adenine Dinucleotide Composition

This example teaches composition and method for a manufacturing process for a nanostructured bioidentical nicotinamide adenine dinucleotide dosage form composition. Phase I is prepared to contain total volume of 270 milliliters of non-lyophilized nicotinamide adenine dinucleotide, Phospholipon 90, propylene glycol, laurocapram, and polysorbate with 11.12% of volume of nicotinamide adenine dinucleotide. Phase II is prepared to contain a 30 ml volume of water and non-phosphate citrate buffer. Phases I and II blend are discharged into a jacketed beaker attached by tubing to a chiller and peristaltic for chilling. The two phases are mixed by a high shear homogenizer for 12 minutes at 8,000 RPM under cooling. The resulting nicotinamide adenine dinucleotide blend is sonicated for 15 minutes by a 40 mm probe with 2500 watts of power at 20 khz with cooling at approx. 50° C. An overhead paddle stirrer at 300 RPM was used to transport fluid uniformly to the probe. Followed by milling on a three-roller mill. The finished product is a 10% nicotinamide adenine dinucleotide concentration of nanostructured bioidentical nicotinamide adenine dinucleotide gel

### EXAMPLE 4

### Formulation of Chemical Drug Delivery Enhancer

This example teaches composition and method for a manufacturing process for a chemical drug delivery enhancer dosage form composition. A total volume of 300 milliliters of ethanol, propylene glycol, laurocapram, cis-oleic acid, dimethyl isosorbide, carbomer 940, d-limonene, I-menthol, and 1.8 cineol are added to 500 ml closed vessel while stirring. Contents are stirred at 1500 RPM and heated to 45° C. for 20 minutes. The finished product is a chemical drug delivery enhancer composition for co-administration with nanostructured bioidentical nicotinamide adenine dinucleotide gel.

### EXAMPLE 5

### Synergistic Effects of Co-Administration

This example teaches compositions and methods of producing synergistic effects between nanostructured bioidentical nicotinamide adenine dinucleotide and a chemical drug delivery enhancer composition from their co-administration. It was tested in-vitro by a one-chambered Franz Diffusion Cell using Strat M pads and PermeaPad membranes to copy skin and gut permeation characteristics. From comparing the flux (the amount of permeant crossing the membrane per time) between a nanostructured bioidentical nicotinamide adenine dinucleotide composition and a 1:1 ratio of bioidentical nicotinamide adenine dinucleotide and chemical drug delivery enhancer composition. After 3 hours HPLC results showed 2055% and 1985% increased transport across the Strat M and PermeaPad membranes from co-administering bioidentical nicotinamide adenine dinucleotide with a chemical drug delivery enhancer composition enhancer.

### Compositions and Methods of Transdermal Nanostructured Nicotinamide Adenine Dinucleotide Drug Delivery

This disclosure teaches compositions and methods of a transdermal nanostructured nicotinamide adenine dinucleotide Drug Delivery System comprised of two formulations: a nanostructured nicotinamide adenine dinucleotide product composition and chemical drug delivery enhancer product composition and co-administering them through the skin's protective stratum corneum barrier and passing through the epidermis to the dermal layers for systemic absorption via the dermal microcirculation.

One embodiment of this disclosure teaches compositions and methods of a transdermal drug delivery system comprised of two formulations: a pharmaceutical, or nutraceutical, or cosmeceutical, or cannabinoid, or phytochemical, or psychedelic drug, or chemical composition, and co-administering them through the skin's protective stratum corneum barrier and passing through the epidermis to the dermal layers for systemic absorption via the dermal microcirculation.

This disclosure teaches compositions methods of a transdermal drug delivery system of nanostructured nicotinamide adenine dinucleotide as a non-invasive alternative to parenteral routes of nicotinamide adenine dinucleotide administration, and circumventing issues such as needle phobia while providing a large surface area of skin and ease of access allowing many placement options on the skin for nicotinamide adenine dinucleotide absorption.

One embodiment of this disclosure teaches compositions and methods of intradermal delivery of nanostructured nicotinamide adenine dinucleotide dosage form drug delivery across the stratum corneum to epidermal and dermal layers as the target of nicotinamide adenine dinucleotide delivery.

One embodiment of this disclosure teaches compositions and methods of topical delivery of a nanostructured nicotinamide adenine dinucleotide to the skin's surface as the target of nicotinamide adenine dinucleotide delivery.

### EXAMPLE 6

### Transdermal Nanostructured Nicotinamide Adenine Dinucleotide and Chemical Drug Delivery Enhancer Co-Administration

Four (4) healthy male and female volunteers between 25 and 75 years of age were recruited. Subjects were not permitted to consume dietary supplements containing niacin, nicotinamide adenine dinucleotide, Nicotinamide Riboside, nicotinamide, or niacin during the three weeks of the trial. Subjects were permitted to use their prescribed medications and supplements other than the above at the same dosage for the duration of the study. A 3 ml syringe was filled with a 10% nicotinamide adenine dinucleotide concentration of stabilized nanostructured bioidentical nicotinamide adenine dinucleotide gel dosage form optimized for transdermal delivery. A 3 ml syringe was filled with a chemical delivery enhancer optimized for nicotinamide adenine dinucleotide transdermal delivery. Subjects dispensed 5 ml from the syringe of nanostructured nicotinamide adenine dinucleotide containing 50 mg of nicotinamide adenine dinucleotide to their left inner wrist/forearm over a 10 square inch area. This was immediately followed by dispensing 0.5 ml of chemical drug delivery enhancer over the nanostructured nicotinamide adenine dinucleotide area of application to the left inner wrist. Subjects rubbed their right wrist to left wrist to disperse the nanostructured nicotinamide adenine dinucleotide gel over their wrist/forearm and maximize the skin area for nicotinamide adenine dinucleotide absorption. This delivered a total dose of 50 mg of nicotinamide adenine dinucleotide for systemic absorption. Subjects immediately performed the same two-step co-administration process of dispensing 0.5 ml nanostructured nicotinamide adenine dinucleotide gel and 0.5 ml chemical delivery enhancer dosage forms to their right inner wrist/forearm for delivering 50 mg of bioidentical nicotinamide adenine dinucleotide to the systemic circulation. This bilateral co-administration method of nicotinamide adenine dinucleotide topical application process to the left and right wrist was repeated three times a day (morning, midday, early evening) for 14 consecutive days, delivering 300 mg day of nicotinamide adenine dinucleotide to the systemic circulation to augment endogenous nicotinamide adenine dinucleotide levels. The subjects whole blood sample was collected on days 1, 7, and 14 via standard venipuncture (contralateral arm to infusion site). 0.1 mL of blood was transferred to a small screw top test tube containing 1 mL of 0.5 M PCM (perchloric acid) solution. All aliquots were immediately frozen and stored at −80° C. awaiting measurement of nicotinamide adenine dinucleotide by liquid chromatography-tandem mass spectrometry (LC-MS/MS).

### Compositions and Methods of Intranasal Nanostructured Nicotinamide Adenine Dinucleotide Drug Delivery

This disclosure teaches compositions and methods of an intranasal nanostructured nicotinamide adenine dinucleotide drug delivery system comprised of two formulations: a nanostructured nicotinamide adenine dinucleotide product and chemical delivery enhancer product composition; and co-administering them through the nasal mucosa to the systemic circulation. Whereas the nicotinamide adenine dinucleotide penetrates through the mucous membranes of the nose and enters the systemic circulation.

One embodiment of this disclosure teaches compositions and methods of a transdermal drug delivery system comprised of two formulations: a pharmaceutical, or nutraceutical, or cosmeceutical, or cannabinoid, or phytochemical, or psychedelic drug, or chemical composition, and a chemical drug delivery enhancer composition and co-administering them through the mucous membranes of the nose into the systemic circulation.

One embodiment of this disclosure teaches compositions and methods of an intranasal nanostructured nicotinamide adenine dinucleotide drug delivery system comprised of two formulations: a nanostructured nicotinamide adenine dinucleotide composition and chemical drug delivery enhancer composition, and co-administering them along the olfactory and trigeminal nerves of the nasal cavity directly into brain for augmenting nicotinamide adenine dinucleotide, or administering nicotinamide adenine dinucleotide therapy.

### EXAMPLE 7

### Intranasal Nanostructured Nicotinamide Adenine dinucleotide and Chemical Drug Delivery Enhancer Co-Administration:

1000 milliliters of 10% nicotinamide adenine dinucleotide concentration of a nanostructured bioidentical nicotinamide adenine dinucleotide gel dosage form were formulated and optimized with a mucoadhesive agent for intranasal delivery. A 15 ml nasal pump spray dispenser bottle with an output volume of 100 MCL per actuation was filled with 15 ml of a 10% nicotinamide adenine dinucleotide concentration of a stabilized nanostructured bioidentical nicotinamide adenine dinucleotide low viscosity fluid dosage form optimized for intraoral delivery. A 15 ml nasal pump spray dispenser bottle with an output volume of 100 mcl per actuation was filled with 15 ml of chemical delivery enhancer optimized for nicotinamide adenine dinucleotide intranasal delivery. Two subjects pump 100 MCL of nanostructured nicotinamide adenine dinucleotide containing 10 mg of nicotinamide adenine dinucleotide into their right nostril. This was immediately followed by co-administering 100 MCL of chemical delivery enhancer into the right nostril by a nasal pump. The subjects repeated the same two-step co-administration process of nanostructured nicotinamide adenine dinucleotide and chemical delivery enhancer into their left nostril. The subjects squeezed nostrils together to improve product mixing and spreading over a larger surface area for absorption into the systemic circulation. The same two-step co-administration process was repeated. This delivers 40 mg of bioidentical nanostructured nicotinamide adenine dinucleotide to the bloodstream. This administration procedure was repeated by test subjects for 14 consecutive days. The subjects whole blood samples were collected on days 1, 7, and 14 via standard venipuncture (contralateral arm to infusion site). 1 mL of blood as was transferred to a small screw top test tube containing 1 mL of 0.5 M PCM (perchloric acid) solution. All the aliquots were immediately frozen and stored at −80° C. awaiting measurement of nicotinamide adenine dinucleotide by liquid chromatography-tandem mass spectrometry (LC-MS/MS).

### Compositions and Methods of Intraoral Nanostructured Nicotinamide Adenine Dinucleotide Drug Delivery

This disclosure teaches compositions and methods of an intraoral nanostructured nicotinamide adenine dinucleotide delivery system comprised of two formulations: a nanostructured nicotinamide adenine dinucleotide composition and a chemical drug delivery enhancer composition and co-administering them through sublingual or buccal mucosa of the oral cavity into the systemic circulation.

One embodiment of this disclosure teaches compositions and methods of an intraoral drug delivery system comprised of two formulations: a pharmaceutical, or nutraceutical, or cosmeceutical, or cannabinoid, or phytochemical, or psychedelic drug, or chemical composition, and a chemical drug delivery enhancer composition and co-administering them through the mucous membranes of the nose into the systemic circulation.

### EXAMPLE 8

### Intraoral Nanostructured Nicotinamide Adenine Dinucleotide and Chemical Drug Delivery Enhancer Co-Administration

A 10% nicotinamide adenine dinucleotide concentration of a nanostructured bioidentical nicotinamide adenine dinucleotide gel dosage form was formulated and optimized with a mucoadhesive agent for intraoral delivery. Three (3) subjects were administered 36 mg NAD+ as nanostructured bioidentical nicotinamide adenine dinucleotide gel to the lower buccal mucosa crease between the cheek and gum by a precision liquid pump device bottle delivering 120 MCL per pump. Each pump delivers 12 mg of bioidentical nicotinamide adenine dinucleotide to the gums. This was immediately followed by co-administering 240 MCL of chemical delivery enhancer to the lower buccal mucosa, by a precision liquid pump device bottle. The subjects were instructed to gently rub their lips to their gum for product mixing and increasing the surface area for nicotinamide adenine dinucleotide absorption into the systemic circulation. The administration protocol was repeated for the upper buccal mucosa for delivering 36 mg of BioNAD+. This administration procedure was repeated by test subjects for 14 consecutive days. Whole blood samples were collected on days 1, 7, and 14 via standard venipuncture (contralateral arm to infusion site). 1 mL of blood was transferred to a small screw top test tube containing 1 mL of 0.5 M PCM (perchloric acid) solution. All the aliquots were immediately frozen and stored −80° C. awaiting measurement of nicotinamide adenine dinucleotide by liquid chromatography-tandem mass spectrometry (LC-MS/MS).

### Compositions and Methods of Nicotinamide Adenine Dinucleotide Oral Drug Delivery

This disclosure teaches compositions and methods of an oral nanostructured nicotinamide adenine dinucleotide drug delivery system comprised of two formulations: a nanostructured nicotinamide adenine dinucleotide composition stabilized for preventing nicotinamide adenine dinucleotide degradation in the gastrointestinal tract and a chemical drug delivery enhancer composition, and co-administering them through the mouth to the gastrointestinal tract, and forming a self-emulsifying drug delivery system in the intestinal tract. Whereas the nicotinamide adenine dinucleotide penetrates through the intestinal lumen and enters the systemic circulation.

One embodiment of this disclosure teaches compositions and methods of an oral drug delivery system comprised of two formulations: a pharmaceutical, or nutraceutical, or cosmeceutical, or cannabinoid, or phytochemical, or psychedelic drug, or chemical composition, and a chemical drug delivery enhancer composition and co-administering them through the mouth to the gastrointestinal tract, and forming a self-emulsifying drug delivery system in the intestinal tract. Whereas the nicotinamide adenine dinucleotide penetrates through the intestinal lumen and enters the systemic circulation.

### EXAMPLE 9

### Oral Nanostructured Nicotinamide Adenine Dinucleotide and Chemical Drug Delivery Enhancer Co-Administration

A 10% nicotinamide adenine dinucleotide concentration of a nanostructured bioidentical nicotinamide adenine dinucleotide dosage stabilized from preventing nicotinamide adenine dinucleotide degradation in the gastrointestinal tract and a chemical delivery enhancer dosage form are formulated and optimized for self-emulsifying in the GI tract. Two (2) subjects swallowed 30 ml nanostructured bioidentical nicotinamide adenine dinucleotide fluid delivered 300 mg of bioidentical nicotinamide adenine dinucleotide. Subject immediately swallowed 200 ml of chemical drug delivery enhancer fluid for self-emulsifying the nicotinamide adenine dinucleotide in their intestinal tract. This administration procedure was repeated by test subject for 14 consecutive days. Subjects whole blood samples were collected on days 1, 7, and 14 via standard venipuncture (contralateral arm to infusion site). 1 mL of blood was transferred into a small screw top test tube containing 1 mL of 0.5 M PCM (perchloric acid) solution. All aliquots were immediately frozen and stored at −80° C. awaiting measurement of nicotinamide adenine dinucleotide by liquid chromatography-tandem mass spectrometry (LC-MS/MS).

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

The description of the present invention has been presented for purposes of illustration and description but is not intended to be exhaustive or limiting of the invention to the form disclosed. Many modifications and variations will be apparent to those of ordinary skill in the art. The embodiment described and shown in the was chosen and described in order to best explain the principles of the invention, the practical application, and to enable others of ordinary skill in the art to understand the invention for various embodiments with various modifications as are suited to the particular use contemplated.

While the compositions and methods that have been described in terms of what are presently considered to be the most practical and preferred embodiments, it is to be understood that the disclosure need not be limited to the disclosed embodiments. It is intended to cover various modifications and similar arrangements included within the spirit and scope of the disclosure, the scope of which should be accorded the broadest interpretation so as to encompass all such modifications and similar structures.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the disclosure both independently and as an overall system and in both method and apparatus modes.

Further, each of the various elements of the disclosure may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the disclosure, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this disclosure is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these disclosure(s), such statements are expressly not to be considered as made by the applicant(s).

Support should be understood to exist to the degree required under new matter laws—including but not limited to United States Patent Law 35 USC 132 or other such laws—to permit the addition of any of the various dependencies or other elements presented under one independent or concept as dependencies or elements under any other independent concept.

To the extent that insubstantial substitutes are made, to the extent that the applicant did not in fact draft any disclosure, method, or composition so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” concept interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms as to afford the applicant the broadest coverage legally permissible.

The description of the present invention has been presented for purposes of illustration and description but is not intended to be exhaustive or limiting of the invention to the form disclosed. Many modifications and variations will be apparent to those of ordinary skill in the art. The embodiment described were chosen and described in order to best explain the principles of the invention, the practical application, and to enable others of ordinary skill in the art to understand the invention for various embodiments with various modifications as are suited to the particular use contemplated.

What is claimed is:

1. A drug delivery system comprising:
nicotinamide adenine dinucleotide or a derivative or precursor thereof; and
chemical drug delivery enhancer composition, wherein the chemical drug delivery enhancer composition comprises an ingredient selected from a group consisting of sulphoxides, polysorbates, azones, pyrrolidones, alcohols, alkanols, surfactants, glycerols, glycols, polyglycols, fatty acids, acidifiers, lipids, phospholipids, terpenes, terpenoids, esters, cyclic oligosaccharides, or a combination thereof.

2. The drug delivery system according to claim 1, wherein the nicotinamide adenine dinucleotide is nanostructured.

3. The drug delivery system according to claim 1, wherein the precursor is selected from a group consisting of nicotinic acid, nicotinamide, nicotinamide riboside, nicotinamide mononucleotide, nicotinic acid riboside or a combination thereof.

4. The drug delivery system according to claim 1, wherein chemical drug delivery enhancer composition comprises glycol.

5. The drug delivery system according to claim 1, wherein the chemical drug delivery enhancer composition comprises:
ethanol: 2%-75%,
propylene glycol: 5%-90%,
laurocapram (1-dodecylazacycloheptan-2-one): 0%-10%
oleic acid: 2%-12%,
polysorbate 20 or polysorbate 60: 1%-15%
2-pyrrolidone: 0%-8%,
isosorbide dimethyl ether: 0%-10%, and
d-limonene, or 1, 8 cineol, or menthol: 2%-12%.

6. A method for delivering a therapeutic agent in blood stream or tissue of a body, the method comprising the steps of:
administering nicotinamide adenine dinucleotide or a derivative or a precursor thereof; and
upon administration, administering a chemical drug delivery enhancer composition.

7. The method according to claim 6, wherein the nicotinamide adenine dinucleotide and the chemical drug delivery enhancer composition are co-administered.

8. The method according to claim 6, wherein the nicotinamide adenine dinucleotide and the chemical drug delivery enhancer composition are administered through a transdermal route.

9. The method according to claim 6, wherein the nicotinamide adenine dinucleotide and the chemical drug delivery enhancer composition are administered through an oral route.

10. The method according to claim 6, wherein the nicotinamide adenine dinucleotide and the chemical drug delivery enhancer composition are administered through a nasal route.

11. The method according to claim 6, wherein the nicotinamide adenine dinucleotide and the chemical drug delivery enhancer composition are administered through a sublingual route.

12. The method according to claim 6, wherein the nicotinamide adenine dinucleotide and the chemical drug delivery enhancer composition are administered through a buccal route.

13. The method according to claim 6, wherein the nicotinamide adenine dinucleotide and the chemical drug delivery enhancer composition are administered through a vaginal route.

14. The method according to claim 6, wherein the nicotinamide adenine dinucleotide and the chemical drug delivery enhancer composition are administered through a rectal route.

15. The method according to claim 6, wherein the nicotinamide adenine dinucleotide and the chemical drug delivery enhancer composition have synergistic effects.

16. The method according to claim 6, wherein the method further comprises the steps of:
administering resveratrol or pterostilbene.

17. A method for treating aging disorders, the method comprising the steps of:
co-administering nicotinamide adenine dinucleotide or a derivative or a precursor thereof on a skin area or into the oral mucosa or into the oral cavity or into the nose; and
a chemical drug delivery enhancer composition.

18. The method according to claim 17, wherein the method is for the therapeutic application of nicotinamide adenine dinucleotide.

19. A drug delivery system comprising:
Co-administering a cannabinoid or psychedelic drug or botanical or nutraceutical cosmeceutical composition; and drug delivery enhancer composition, wherein the chemical drug delivery enhancer composition comprises an ingredient selected from a group consisting of sulphoxides, polysorbates, azones, pyrrolidones, alcohols, alkanols, surfactants, glycerols, glycols, polyglycols, fatty acids, acidifiers, lipids, phospholipids, terpenes, terpenoids, esters, cyclic oligosaccharides, or a combination thereof.

20. The drug delivery system according to claim 19, wherein the nicotinamide adenine dinucleotide is nanostructured.
